(19) United States
(12) Patent Application Publication
(10) Pub. No.: US 2008/0045510 A1
(43) Pub. Date: Feb. 21, 2008

(54) Succinate salts of 6-methoxy-8-[4-(1-(5-fluoro)-quinolin-8-yl-piperidin-4-yl)-piperazin-1-yl]-quinoline and crystalline forms thereof

(75) Inventors: Jessica K. Liang, Suffern, NY (US); Shuimei Wang, Highland Mills, NY (US)

Correspondence Address: FISH & RICHARDSON P.C., P.O BOX 1022, MINNEAPOLIS, MN, 55440-1022, US

(73) Assignee: Wyeth, Madison, NJ (US)

(21) Appl. No.: 11/811,022

(22) Filed: Jun. 8, 2007

Related U.S. Application Data
(60) Provisional application No. 60/812,384, filed on Jun. 9, 2006.

(51) Int. Cl. A61K 31/496; A61K 31/506; A61K 31/551; A61K 31/5513; A61P 15/00; A61P 25/16; A61P 25/18; A61P 25/22; A61P 25/28; A61P 25/30; A61P 3/04; C07D 401/14

(52) U.S. Cl. 514/220; 514/221; 514/252.18; 514/253.06; 544/363

(57) ABSTRACT

The present invention relates to succinic acid salt forms of the 5-HT_{1A} binding agent 6-methoxy-8-[4-(1-(5-fluoro)-quinolin-8-yl-piperidin-4-yl)-piperazin-1-yl]-quinoline, as well as crystalline forms thereof, pharmaceutical compositions thereof, and methods of use thereof.

### CROSS REFERENCE TO RELATED APPLICATIONS

This application claims the benefit of U.S. Provisional Pat. App. Ser. No. 60/812,384, filed Jun. 9, 2006, the disclosure of which is incorporated herein by reference in its entirety.

### FIELD OF THE INVENTION

The present invention relates to succinic acid salts of the 5-HT_{1A} binding agent 6-methoxy-8-[4-(1-(5-fluoro)-quinolin-8-yl-piperidin-4-yl)-piperazin-1-yl]-quinoline, as well as crystalline forms thereof, pharmaceutical compositions thereof, and methods of use thereof.

### BACKGROUND OF THE INVENTION

N-Aryl-piperazine derivatives are known to bind to 5-HT_{1A} receptors and are useful as pharmaceutical agents for the treatment of various central nervous system (CNS) disorders such as cognitive disorders, anxiety disorders, and depression. See, e.g., Childers, et al., J. Med. Chem., 2005, 48, 3467; and U.S. Pat. Nos. 6,465,482; 6,127,357; 6,469,007; and 6,586,436, as well as WO 97/03982. Among these, certain N-aryl-piperazine-piperidine compounds, including 6-methoxy-8-[4-(1-(5-fluoro)-quinolin-8-yl-piperidin-4-yl)-piperazin-1-yl]-quinoline (see Formula I), which is described in WO 2006/135839 have been found to modulate activity of the 5-HT_{1A} receptor and are useful, for example, for enhancing cognition, treating anxiety, and treating depression, among other CNS disorders.

Drug compounds are typically combined with other pharmaceutically acceptable ingredients to form compositions suitable for a desired mode of administration. Solid formulations often require that the drug compound have workable solid state characteristics such as stability to heat and humidity, ease of handling, and other characteristics that facilitate preparation of solid dosage forms. At the same time, good water solubility, which often translates to good bioavailability, is also desired. Accordingly, there is an ongoing need for more stable and more soluble solid forms of existing drug molecules. The salt and crystalline forms of 6-methoxy-8-[4-(1-(5-fluoro)-quinolin-8-yl-piperidin-4-yl)-piperazin-1-yl]-quinoline described herein are directed toward this end.

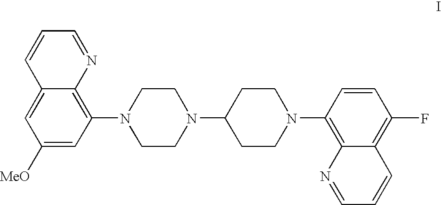

### SUMMARY OF THE INVENTION

The present invention provides a succinate salt, including a trisuccinate salt, of 6-methoxy-8-[4-(1-(5-fluoro)-quinolin-8-yl-piperidin-4-yl)-piperazin-1-yl]-quinoline.

The present invention further provides a crystalline form of 6-methoxy-8-[4-(1-(5-fluoro)-quinolin-8-yl-piperidin-4-yl)-piperazin-1-yl]-quinoline trisuccinate designated as Form A.

The present invention further provides a crystalline form of 6-methoxy-8-[4-(1-(5-fluoro)-quinolin-8-yl-piperidin-4-yl)-piperazin-1-yl]-quinoline trisuccinate designated as Form B.

The present invention further provides a crystalline form of 6-methoxy-8-[4-(1-(5-fluoro)-quinolin-8-yl-piperidin-4-yl)-piperazin-1-yl]-quinoline trisuccinate designated as Form C.

The present invention further provides a crystalline form of 6-methoxy-8-[4-(1-(5-fluoro)-quinolin-8-yl-piperidin-4-yl)-piperazin-1-yl]-quinoline trisuccinate designated as Form D.

The present invention further provides compositions comprising the salts and crystalline forms described herein.

The present invention further provides processes for the preparation of the salts and crystalline forms described herein.

The present invention further provides salts and crystalline forms prepared by the processes of preparation described herein.

The present invention further provides methods of treating 5-HT_{1A} associated diseases by administering to a patient a therapeutically effective amount of a salt or crystalline form described herein, or composition thereof.

### BRIEF DESCRIPTION OF THE DRAWINGS

FIG. 1 depicts an XRPD pattern consistent with Form A.

FIG. 2 depicts an XRPD pattern consistent with Form B.

FIG. 3 depicts an XRPD pattern consistent with Form C.

FIG. 4 depicts an XRPD pattern consistent with Form D.

FIG. 5 depicts DSC and TGA data consistent with Form A.

FIG. 6 depicts DSC data consistent with Form B.

FIG. 7 depicts DSC data consistent with Form C.

FIG. 8 depicts DSC data consistent with Form D.

FIG. 9 depicts TGA data consistent with Form D.

### DETAILED DESCRIPTION

Succinate Salts

The present invention provides, inter alia, succinic acid [CH₂(O)(OH)—CH₂C(O)(OH)] salts, including the trisuccinate salt, of the compound 6-methoxy-8-[4-(1-(5-fluoro)-quinolin-8-yl-piperidin-4-yl)-piperazin-1-yl]-quinoline (see Formula I above) which can modulate the 5-HT_{1A} receptor and is useful in the treatment of CNS disorders. The salts of the invention can be crystalline, amorphous, or a combination thereof. In some embodiments, the salt is a trisuccinate salt which is substantially crystalline. In further embodiments, the crystalline trisuccinate salt is characterized as having a particular crystalline form, such as any of those described herein.

The trisuccinate salt of 6-methoxy-8-[4-(1-(5-fluoro)-quinolin-8-yl-piperidin-4-yl)-piperazin-1-yl]-quinoline possesses numerous advantages over the free base form. For example, the free base has relatively poor solubility in aqueous media (about 0.4 μg/mL) even in the presence of surfactants, indicating potential poor bioavailability. In contrast, the trisuccinate salt is much more soluble in water (1.2 mg/mL) than the free base, and has improved bioavailability over the free base. Other advantages of the trisuccinate salt include its crystallinity which aids in preparation of substantially pure API and facilitates handling.

Crystalline Forms

As used herein, “crystalline form” is meant to refer to a certain lattice configuration of a crystalline substance. Different crystalline forms of the same substance (e.g., 6-methoxy-8-[4-(1-(5-fluoro)-quinolin-8-yl-piperidin-4-yl)-piperazin-1-yl]-quinoline trisuccinate) typically have different crystalline lattices (e.g., unit cells), typically have different physical properties attributed to their different crystalline lattices, and in some instances, have different water or solvent content. The different crystalline lattices can be identified by solid state characterization methods such as by X-ray powder diffraction (XRPD). Other characterization methods such as differential scanning calorimetry (DSC), thermogravimetric analysis (TGA), dynamic vapor sorption (DVS), and the like further help identify the crystalline form as well as help determine stability and solvent/water content.

Different crystalline forms of a particular substance, such as a salt of the invention, can include both anhydrous forms of that substance and solvated/hydrated forms of that substance, where each of the anhydrous forms and solvated/hydrated forms are distinguished from each other by different XRPD patterns, thereby signifying different crystalline lattices. In some instances, a single crystalline form (e.g., identified by a unique XRPD pattern) can have variable water or solvent content, where the lattice remains substantially unchanged (as does the XRPD pattern) despite the compositional variation with respect to water and/or solvent.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the machine or the settings (for example, whether a Ni filter is used or not). As used herein, the term “peak” refers to a reflection having a relative height/intensity of at least about 4% of the maximum peak height/intensity. Moreover, instrument variation and other factors can affect the 2-theta values. Thus, peak assignments, such as those reported herein, can vary by plus or minus about 0.20 (2-theta), and the term “substantially” as used in the context of XRPD herein is meant to encompass the above-mentioned variations.

In the same way, temperature readings in connection with DSC, TGA, or other thermal experiments can vary about ±3° C. depending on the instrument, particular settings, sample preparation, etc. Accordingly, a crystalline form reported herein having a DSC thermogram “substantially” as shown in any of the Figures is understood to accommodate such variation.

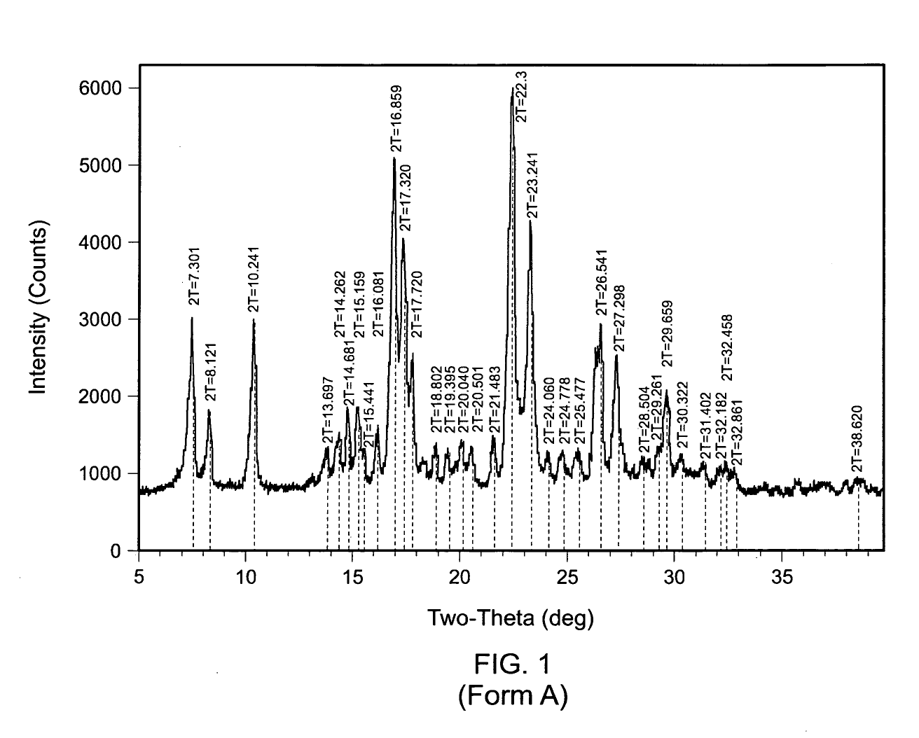

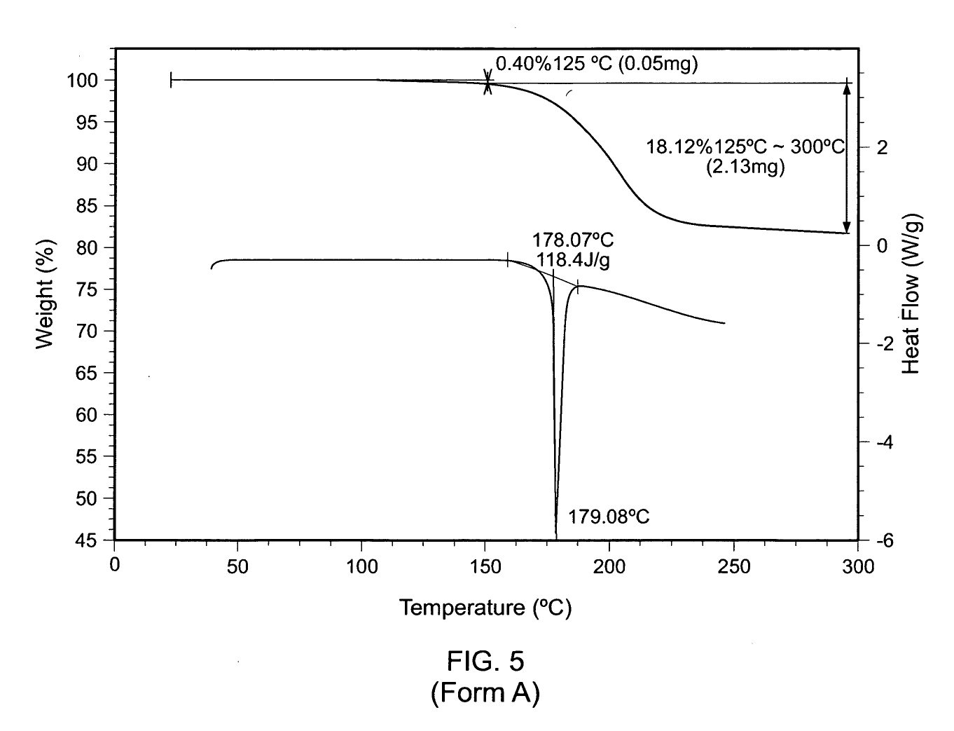

The present invention provides four crystalline forms of 6-methoxy-8-[4-(1-(5-fluoro)-quinolin-8-yl-piperidin-4-yl)-piperazin-1-yl]-quinoline trisuccinate. A first crystalline form, designated Form A, is believed to be substantially anhydrous and substantially non-hygroscopic. Form A is characterized according to the XRPD pattern of FIG. 1 (see Table 1 for peak data) as well as the DSC and TGA data provided in FIG. 5 (see Table 2). The DSC trace showed a single endotherm centered at about 179° C. which is attributed to a melt event. The TGA data showed essentially negligible weight loss up to about 125° C. and then greater weight loss from about 125° C. to about 300° C. which is believed to correspond to melt and decomposition events. Form A was found to be stable at room temperature and high relative humidity, but can convert to Form D (putatively a hydrate) upon dispersion in water for a few minutes in ambient conditions.

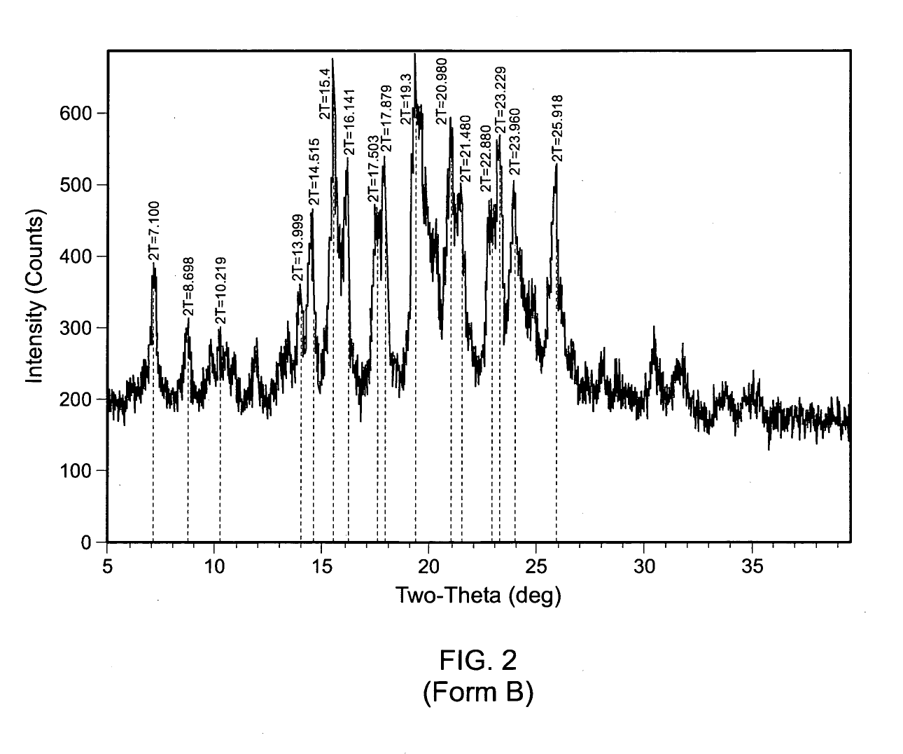

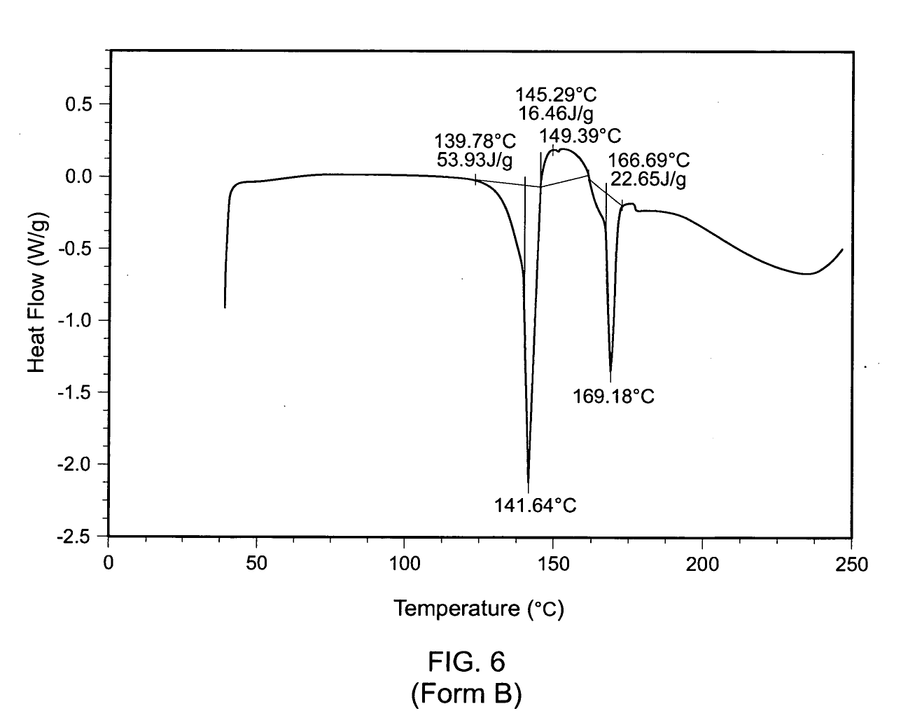

A second crystalline form, designated Form B, is believed to be substantially anhydrous. Form B is characterized according to the XRPD pattern of FIG. 2 (see Table 1 for peak data) as well as the DSC data provided in FIG. 6 (see Table 2). The DSC trace showed an endotherm centered at about 142° C., an exotherm centered at about 149° C., and a second endotherm centered at about 169° C. which is attributed to a melt/decomposition event. Form B appeared to convert to Form A when slurried for two hours in a mixture of THF and heptane in an ice/water bath.

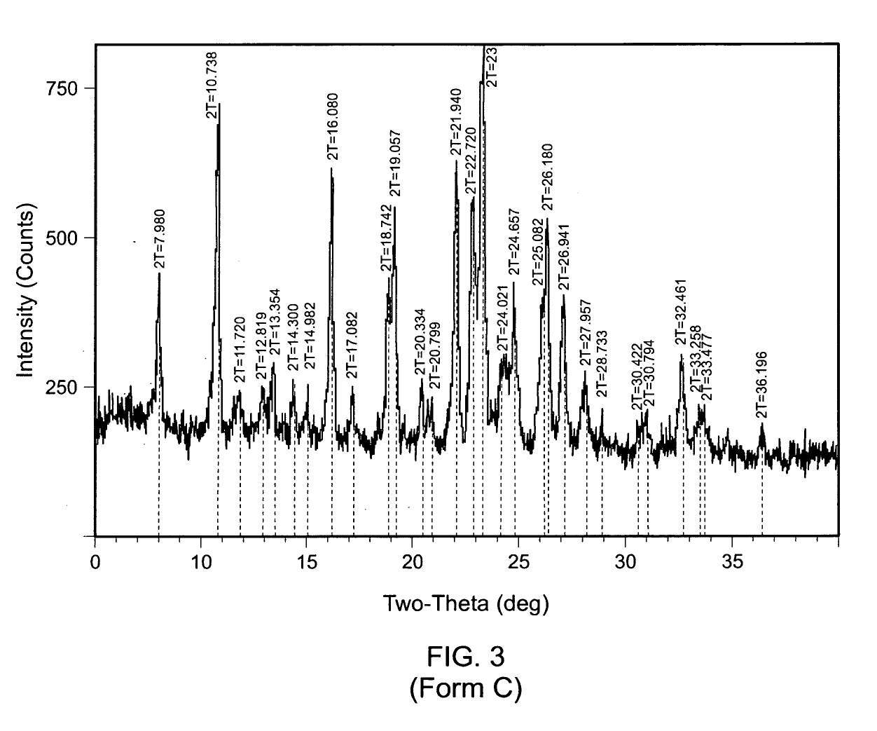

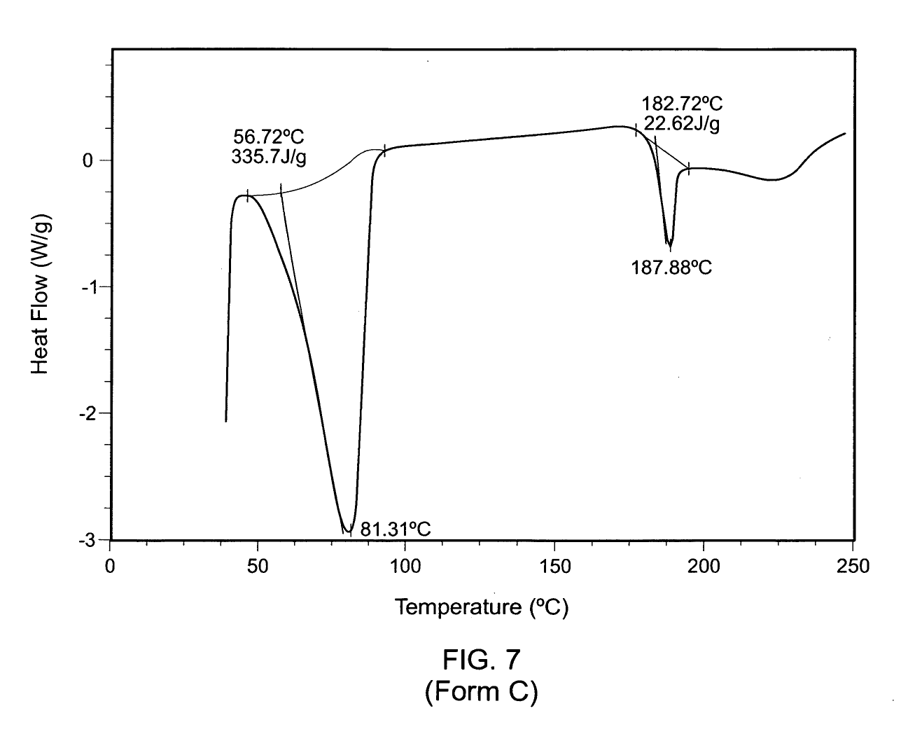

A third crystalline form, designated Form C, is believed to be a hydrate. Form C is characterized according to the XRPD pattern of FIG. 3 (see Table 1 for peak data) as well as the DSC data provided in FIG. 7 (see Table 2). The DSC trace showed a first endotherm centered at about 81° C. (possibly a dehydration event) and a second endotherm centered at about 188° C.

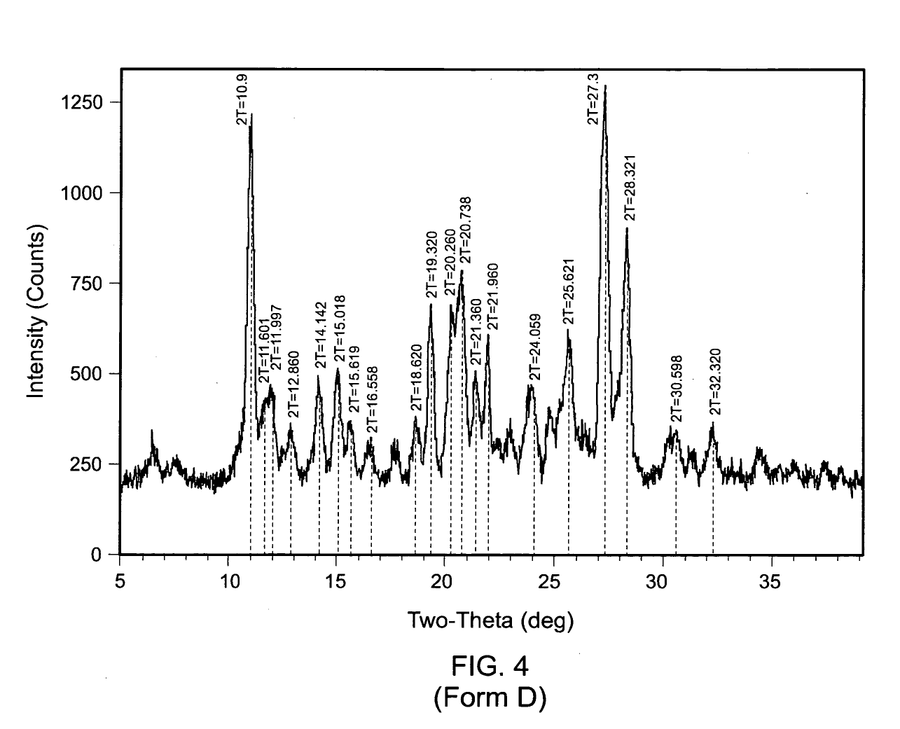

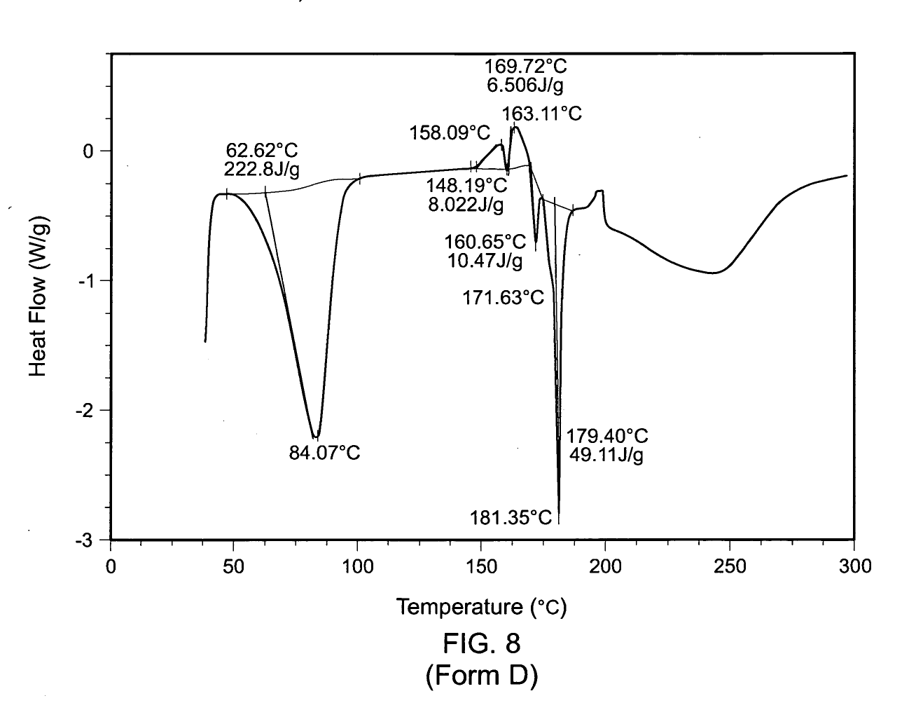

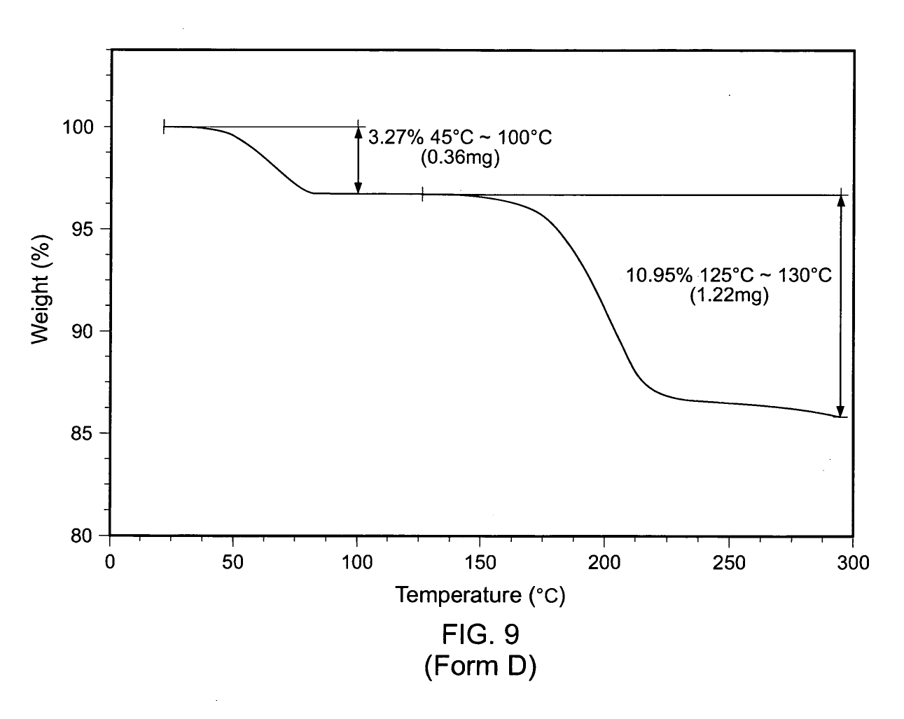

A fourth crystalline form, designated Form D, is believed to be a hydrate. Form D is characterized according to the XRPD pattern of FIG. 4 (see Table 1 for peak data) as well as the DSC data provided in FIG. 8 and the TGA data provided in FIG. 9 (see Table 2). The DSC trace showed a first endotherm centered at about 84° C. which is believed to correspond to a dehydration event. Two exotherms were observed at about 158° C. and about 163° C. Two additional endotherms were observed at about 172° C. and about 181° C., attributed to decomposition. The TGA data showed a weight loss of about 3.27% from about 45° C. to about 100° C. which corresponded to about 1.5 moles of water. Additional weight loss was observed at temperatures higher than 150° C. which may be due to decomposition.

XRPD peak and intensity data acquired for each of the four crystalline forms are provide below in Table 1. Instrument and collection parameters are provided below in the Examples. Intensities are provided as relative intensities such that +++ represents an intensity that is equal to or greater than 50% of the maximum intensity; ++ represents an intensity that is equal to or greater than 25% of the maximum intensity but less than 50% of the maximum intensity; and +represents an intensity that is less than 25% of the maximum intensity.

TABLE 1

| Form A | | Form B | | Form C | | Form D | |
|---|---|---|---|---|---|---|---|
| 2-theta | Intensity | 2-theta | Intensity | 2-theta | Intensity | 2-theta | Intensity |
| 7.3 | ++ | 7.1 | ++ | 8.0 | ++ | 11.0 | +++ |
| 8.1 | + | 8.7 | ++ | 10.7 | +++ | 11.6 | + |
| 10.2 | ++ | 10.2 | + | 11.7 | + | 12.0 | + |
| 13.7 | + | 14.0 | ++ | 12.8 | + | 12.9 | + |
| 14.3 | + | 14.5 | +++ | 13.4 | + | 14.1 | ++ |
| 14.7 | + | 15.5 | +++ | 14.3 | + | 15.0 | ++ |
| 15.2 | + | 16.1 | +++ | 15.0 | + | 15.6 | + |
| 15.4 | + | 17.5 | +++ | 16.1 | +++ | 16.6 | + |
| 16.1 | + | 17.9 | +++ | 17.1 | + | 18.6 | + |
| 16.9 | +++ | 19.3 | +++ | 18.7 | ++ | 19.3 | ++ |
| 17.3 | +++ | 21.0 | +++ | 19.1 | +++ | 20.3 | ++ |
| 17.7 | ++ | 21.5 | +++ | 20.3 | + | 20.7 | +++ |
| 18.8 | + | 22.9 | ++ | 20.8 | + | 21.4 | + |
| 19.4 | + | 23.3 | +++ | 21.9 | +++ | 22.0 | ++ |
| 20.0 | + | 24.0 | ++ | 22.7 | +++ | 24.1 | + |
| 20.5 | + | 25.9 | +++ | 23.1 | +++ | 25.6 | ++ |
| 21.5 | + | | | 24.0 | + | 27.3 | +++ |
| 22.4 | +++ | | | 24.7 | ++ | 28.3 | +++ |
| 23.2 | +++ | | | 26.0 | ++ | 30.6 | + |
| 24.1 | + | | | 26.2 | ++ | 32.3 | + |
| 24.8 | + | | | 26.9 | ++ | | |
| 25.5 | + | | | 28.0 | + | | |
| 26.5 | ++ | | | 28.7 | + | | |
| 27.3 | ++ | | | 30.4 | + | | |
| 28.5 | + | | | 30.8 | + | | |
| 29.3 | + | | | 32.5 | + | | |
| 29.7 | + | | | 33.3 | + | | |
| 30.3 | + | | | 33.5 | + | | |
| 31.4 | + | | | 36.2 | + | | |
| 32.2 | + | | | | | | |
| 32.5 | + | | | | | | |
| 32.9 | + | | | | | | |
| 38.6 | + | | | | | | |

Select physical properties for each of the four crystalline forms described herein are compared in Table 2.

TABLE 2

| | Form A | Form B | Form C | Form D |
|---|---|---|---|---|
| XRPD select peaks | 7.3, 8.1, 10.2, | 7.1, 8.7, 14.5, 16.1, | 8.0, 10.7, 16.1, | 11.0, 14.1, 15.0, |
| (° 2-theta) | 16.9, 17.3, 17.7, | 17.9, 21.0, 23.3 | 18.7, 19.1, 21.9, | 19.3, 20.3, 20.7, |
| | 22.4, 23.2 | | 22.7, 23.1, 24.7 | 27.3, 28.3 |
| DSC | 179 (endo) | 143 (endo) | 81 (endo) | 84 (endo) |
| (° C.) | | 149 (exo) | 188 (endo) | 158 (exo) |
| | | 169 (endo) | | 163 (exo) |
| | | | | 172 (endo) |
| | | | | 181 (endo) |
| TGA | negligible weight | — | — | 3.27% weight |
| | loss up to 125° C. | | | loss from 45° C. |
| | significant weight | | | to 100° C. |
| | loss between 125° | | | significant |
| | C. and 300° C. | | | weight loss above |
| | (melt/decomp) | | | 150° C. |

Advantages of each of the crystalline forms is readily apparent. For example, the non-hygroscopic properties of Form A coupled with apparent thermodynamic stability would impart a relatively long shelf life to solid pharmaceutical formulations made with this crystalline form. Form B as a meta-stable form would be expected to have improved solubility over Form A and therefore show better bioavailability. Forms C and D as hydrates would have the advantage of being able to be prepared under conditions that were not rigorously water-free, and allow the use of less hazardous aqueous solvents during preparation.

In some embodiments, the present invention provides a crystalline form (Form A) of 6-methoxy-8-[4-(1-(5-fluoro)-quinolin-8-yl-piperidin-4-yl)-piperazin-1-yl]-quinoline trisuccinate having an X-ray powder diffraction pattern comprising characteristic peaks, in terms of 2θ (°), at about 8.1 and about 22.4. In some embodiments, the XRPD pattern further comprises a characteristic peak at about 10.2. In yet further embodiments, the XRPD pattern further comprises a characteristic peak at about 16.9.

In some embodiments, the present invention provides a crystalline form (Form A) of 6-methoxy-8-[4-(1-(5-fluoro)-quinolin-8-yl-piperidin-4-yl)-piperazin-1-yl]-quinoline trisuccinate having an X-ray powder diffraction pattern comprising a characteristic peak, in terms of 2θ (°), of about 8.1 and at least three characteristic peaks, in terms of 20, selected from about 7.3, about 10.2, about 16.9, about 17.3, about 17.7, about 22.4, about 23.2, about 26.5, about 27.3, and about 29.7.

In some embodiments, the present invention provides a crystalline form (Form A) of 6-methoxy-8-[4-(1-(5-fluoro)-quinolin-8-yl-piperidin-4-yl)-piperazin-1-yl]-quinoline trisuccinate having an X-ray powder diffraction pattern substantially as shown in FIG. 1.

In some embodiments, the present invention provides a crystalline form (Form A) of having a DSC thermogram which is characterized by an endothermic peak at about 179° C. In further embodiments, the crystalline form has a DSC thermogram substantially as shown in FIG. 5. In yet further embodiments, the crystalline form has a TGA profile substantially as shown in FIG. 5.

In some embodiments, the present invention provides a crystalline form (Form B) of 6-methoxy-8-[4-(1-(5-fluoro)-quinolin-8-yl-piperidin-4-yl)-piperazin-1-yl]-quinoline trisuccinate having an X-ray powder diffraction pattern comprising characteristic peaks, in terms of 2θ (°), at about 7.1 and about 21.0. In some embodiments, the XRPD pattern further comprises a characteristic peak at about 15.5. In further embodiments, the XRPD pattern further comprises a characteristic peak at about 25.9.

In some embodiments, the present invention provides a crystalline form (Form B) of 6-methoxy-8-[4-(1-(5-fluoro)-quinolin-8-yl-piperidin-4-yl)-piperazin-1-yl]-quinoline trisuccinate having an X-ray powder diffraction pattern comprising a characteristic peak, in terms of 2θ, of about 7.1 and at least three characteristic peaks, in terms of 2θ, selected from about 8.7, about 14.5, about 15.5, about 16.1, about 17.9, about 19.3, about 21.0, about 23.3, about 24.0, and about 25.9.

In some embodiments, the present invention provides a crystalline form (Form B) of 6-methoxy-8-[4-(1-(5-fluoro)-quinolin-8-yl-piperidin-4-yl)-piperazin-1-yl]-quinoline trisuccinate having an X-ray powder diffraction pattern substantially as shown in FIG. 2.

In some embodiments, the present invention provides a crystalline form having a DSC thermogram substantially as shown in FIG. 6.

In some embodiments, the present invention provides a crystalline form (Form C) of 6-methoxy-8-[4-(1-(5-fluoro)-quinolin-8-yl-piperidin-4-yl)-piperazin-1-yl]-quinoline trisuccinate having an X-ray powder diffraction pattern comprising characteristic peaks, in terms of 2θ (°), at about 8.0 and about 10.7. In some embodiments, the XRPD pattern further comprises a characteristic peak at about 16.1. In yet further embodiments, the XRPD pattern further comprises a characteristic peak at about 23.1.

In some embodiments, the present invention provides a crystalline form (Form C) of 6-methoxy-8-[4-(1-(5-fluoro)-quinolin-8-yl-piperidin-4-yl)-piperazin-1-yl]-quinoline trisuccinate having an X-ray powder diffraction pattern comprising a characteristic peak, in terms of 2θ, of about 10.7 and at least three characteristic peaks, in terms of 2θ, selected from about 8.0, about 16.1, about 18.7, about 19.1, about 21.9, about 22.7, about 23.1, about 24.7, about 26.0, about 26.3, about 26.9, and about 32.5.

In some embodiments, the present invention provides a crystalline form (Form C) of 6-methoxy-8-[4-(1-(5-fluoro)-quinolin-8-yl-piperidin-4-yl)-piperazin-1-yl]-quinoline trisuccinate having an X-ray powder diffraction pattern substantially as shown in FIG. 3.

In some embodiments, the present invention provides a crystalline form having a DSC thermogram substantially as shown in FIG. 7.

In some embodiments, the present invention provides a crystalline form (Form D) of 6-methoxy-8-[4-(1-(5-fluoro)-quinolin-8-yl-piperidin-4-yl)-piperazin-1-yl]-quinoline trisuccinate having an X-ray powder diffraction pattern comprising characteristic peaks, in terms of 2θ (°), at about 11.0 and about 27.3. In further embodiments, the XRPD pattern further comprises a characteristic peak at about 28.3. In further embodiments, the XRPD pattern further comprises a characteristic peak at about 20.7.

In some embodiments, the present invention provides a crystalline form (Form D) of 6-methoxy-8-[4-(1-(5-fluoro)-quinolin-8-yl-piperidin-4-yl)-piperazin-1-yl]-quinoline trisuccinate having an X-ray powder diffraction pattern comprising a characteristic peak, in terms of 2θ (°), of about 11.0 and at least three characteristic peaks, in terms of 2θ, selected from about 14.1, about 15.0, about 19.3, about 20.3, about 20.7, about 22.0, about 25.6, about 27.3, about 28.3, and about 32.3.

In some embodiments, the present invention provides a crystalline form (Form D) of 6-methoxy-8-[4-(1-(5-fluoro)-quinolin-8-yl-piperidin-4-yl)-piperazin-1-yl]-quinoline trisuccinate having an X-ray powder diffraction pattern substantially as shown in FIG. 4.

In some embodiments, the present invention provides a crystalline form having a DSC thermogram substantially as shown in FIG. 8.

In some embodiments, the present invention provides a crystalline form having a TGA profile substantially as shown in FIG. 9.

Compositions

The present invention further provides compositions containing a succinate salt or crystalline form of the invention and one or more other ingredients. In some embodiments, the composition contains at least about 50%, at least about 70%, at least about 80%, at least about 90%, at least about 95%, at least about 97%, at least about 98.0%, at least about 98.1%, at least about 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9% by weight of the salt 6-methoxy-8-[4-(1-(5-fluoro)-quinolin-8-yl-piperidin-4-yl)-piperazin-1-yl]-quinoline trisuccinate. In some embodiments, the composition is a pharmaceutical composition which contains at least one active pharmaceutical ingredient which is 6-methoxy-8-[4-(1-(5-fluoro)-quinolin-8-yl-piperidin-4-yl)-piperazin-1-yl]-quinoline trisuccinate and at least one pharmaceutically acceptable carrier.

In some embodiments, the composition contains at least about 50%, at least about 70%, at least about 80%, at least about 90%, at least about 95%, at least about 97%, at least about 98.0%, at least about 98.1%, at least about 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9% by weight of 6-methoxy-8-[4-(1-(5-fluoro)-quinolin-8-yl-piperidin-4-yl)-piperazin-1-yl]-quinoline trisuccinate having crystalline Form A, Form B, Form C, or Form D. In further embodiments, crystalline Form A, Form B, Form C, or Form D constitutes at least about 50%, at least about 70%, at least about 80%, at least about 90%, at least about 95%, at least about 97%, at least about 98.0%, at least about 98.1%, at least about 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9% by weight of total 6-methoxy-8-[4-(1-(5-fluoro)-quinolin-8-yl-piperidin-4-yl)-piperazin-1-yl]-quinoline trisuccinate present in the composition.

In some embodiments, the composition is a pharmaceutical composition which contains at least one active pharmaceutical ingredient which is 6-methoxy-8-[4-(1-(5-fluoro)-quinolin-8-yl-piperidin-4-yl)-piperazin-1-yl]-quinoline trisuccinate having Form A, Form B, Form C, or Form D and at least one pharmaceutically acceptable carrier. In further embodiments, the composition is a pharmaceutical composition which contains at least one active pharmaceutical ingredient which is 6-methoxy-8-[4-(1-(5-fluoro)-quinolin-8-yl-piperidin-4-yl)-piperazin-1-yl]-quinoline trisuccinate having Form A and at least one pharmaceutically acceptable carrier. In some embodiments, the pharmaceutical composition is suitable for oral administration. In some embodiments, the composition is provided in the form of a sustained release dosage form.

In some embodiments, the compositions can contain mixtures of Form A, Form B, Form C, Form D, or any subset thereof. The presence of different crystalline forms in a composition can be assessed by a suitable spectroscopic method such as X-ray powder diffraction.

The pharmaceutical composition of the invention can further contain one or more additional active pharmaceutical ingredients (APIs). For example, the compositions can further include one or more antidepressants or anti-anxiety agents. Example classes of antidepressants include norepinephrine reuptake inhibitors, selected serotonin reuptake inhibitors (SSRIs), NK-1 receptor antagonists, monoamine oxidase inhibitors (MAOs), reversible inhibitors of monoamine oxidase (RIMAs), serotonin and noradrenaline reuptake inhibitors (SNRIs), corticotrophin releasing factor (CRF) antagonists, alpha-adrenoreceptor antagonists, and atypical antidepressants. Example suitable norepinephrine reuptake inhibitors include tertiary amines tricyclics and secondary amine tricyclics. Suitable tertiary amine tricyclics and secondary amine tricyclics include amitriptyline, clomipramine, doxepin, imipramine, trimipramine, dothiepin, butripyline, iprindole, lofepramine, nortryptline, protriptline, amoxapine, desipramine and maprotiline. Suitable selective serotonin reuptake inhibitors include fluoxetine, fluvoxaine, paroxetine and sertraline. Examples of monoamine oxidase inhibitors include isocarboxazid, pheneizine, and tranylcyclopramine. Suitable reversible inhibitors of monoamine oxidase include moclobemide. Suitable serotonin and noradrenaline reuptake inhibitors include venlafaxine. Suitable CRF antagonists include those described in WO 94/13643, WO 94/13644, WO 94/13661, WO 94/13676, and WO 94/13677. Suitable atypical antidepressants include bupropion, lithium, nefazodone, trazodone and viloxazine. Suitable NK-1 receptor antagonists include those described in WO 01/77100.

Anti-anxiety agents that can be used in combination with the salts and crystalline forms of the invention include benzodiazepines and serotonin 1A (5-HT_{1A}) agonists or antagonists, including partial agonists, and CRF antagonists. Suitable benzodiazepines include alprazolam, chlordiazepoxide, clonazepam, chlorazepate, diazepam, halazepam, lorazepam, oxazepam, and prazedpam. Suitable 5-HT_{1A} receptor agonists or antagonists include buspirone, flesinoxan, gepirone and ipsapirone.

In some embodiments, the pharmaceutical composition is provided in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form.

Pharmaceutical compositions can be prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those carriers that are compatible with the other ingredients in the formulation and are biologically acceptable.

Preparation of Salt and Crystalline Forms

The succinate salts of the invention can be prepared according to routine methods. For example, the free base compound 6-methoxy-8-[4-(1-(5-fluoro)-quinolin-8-yl-piperidin-4-yl)-piperazin-1-yl]-quinoline can be combined with an appropriate amount of succinic acid to form a succinate salt. Depending on the desired salt, different molar ratios of free base to acid can be used. For example, a free base:acid molar ratio of about 1:1 can be used to generate a monosuccinate salt; a free base:acid molar ratio of about 1:2 can be used to generate a disuccinate salt; a free base:acid molar ratio of about 1:3 can be used to generate a trisuccinate salt. The combining can be carried out by any suitable method. For example, a solution of the free base in a suitable organic solvent can be combined with a solution of the succinic acid in a different organic solvent. Because the free base is typically less soluble in aqueous and polar solvents than the succinate salt forms of the free base, solvents can be chosen such that the final solvent or solvent mixture resulting from the combination of separate solutions of free base and succinic acid is a poor solvent for the final succinic acid salt of 6-methoxy-8-[4-(1-(5-fluoro)-quinolin-8-yl-piperidin-4-yl)-piperazin-1-yl]-quinoline, thereby aiding in the precipitation, separation, and/or purification of the solid salt product.

Crystalline Form A can be prepared by precipitation from a solution of 6-methoxy-8-[4-(1-(5-fluoro)-quinolin-8-yl-piperidin-4-yl)-piperazin-1-yl]-quinoline trisuccinate in organic solvent which is substantially free of water. Suitable organic solvents include, for example, aliphatics (e.g., pentane, hexanes, heptane, etc.), halogenated aliphatics (e.g., dichloromethane), aromatics (e.g., benzene, toluene, pyridine, chlorobenzene, etc.), ketones (e.g., acetone, 2-butanone, etc.), ethers (diethyl ether, tetrahydrofuran, etc.), alcohols (e.g., methanol, ethanol, butanol, etc.), and the like and mixtures thereof. In some embodiments, the organic solvent contains dichloromethane, acetone, tetrahydrofuran, or mixture thereof. The term “substantially free of water” is meant to refer to organic solvents containing about 3% water or less, about 2% water or less, about 1% water or less, or about 0.5% water or less, on a volume basis. In some embodiments, the solvent contains about 0.5% or less of water by volume. Precipitation can be induced by any of many routine methods including lowering the temperature of the solution, concentrating the solution by evaporation (e.g., under air, under gas flow, or under vacuum), seeding, addition of antisolvent, or combination of any of these techniques. In some embodiments, precipitation is induced by addition of antisolvent (e.g., heptane or other aliphatic) and/or reduction of solution temperature. In some embodiments, immediate precipitation can result in a crystalline form other than Form A; however, passage of sufficient time in the solution from which the material precipitated can result in formation of Form A due to its putative thermodynamic stability.

Crystalline Form B can be prepared by precipitation from a solution of 6-methoxy-8-[4-(1-(5-fluoro)-quinolin-8-yl-piperidin-4-yl)-piperazin-1-yl]-quinoline trisuccinate in organic solvent which is substantially free of water. Suitable organic solvents include those that are listed above. In some embodiments, the solvent contains about 0.5% or less of water by volume. In some embodiments, the organic solvent contains tetrahydrofuran. Precipitation can be induced by any of the methods described above. In some embodiments, precipitation is induced by addition of antisolvent (e.g., heptane or other aliphatic) and/or reduction of solution temperature. Form B is typically isolated prior to its conversion to another crystalline form, such as Form A. In some embodiments, Form B is immediately isolated from the solution in which it was formed.

Crystalline Form C can be prepared by combining Form A with an aqueous solvent. In some embodiments, Form A can be slurried in water for a time sufficient to generate Form C. Seeding with Form C can further aid in preparations of that crystalline form.

Crystalline Form D can be prepared by combining Form A with an aqueous solvent. In some embodiments, Form A can be slurried in a mixture containing water and alcohol for a time sufficient to form crystalline Form D. In some embodiments, the alcohol is ethanol. Seeding with Form D can further aid in preparations of that crystalline form.

The present invention further provides crystalline Forms A, B, C, and D prepared by any of the methods described herein.

Compositions

The present invention further provides compositions containing a salt or crystalline form of the invention and one or more other ingredients. In some embodiments, the composition contains at least about 50%, at least about 70%, at least about 80%, at least about 90%, at least about 95%, at least about 97%, at least about 98.0%, at least about 98.1%, at least about 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9% by weight of a succinic acid salt 6-methoxy-8-[4-(1-(5-fluoro)-quinolin-8-yl-piperidin-4-yl)-piperazin-1-yl]-quinoline. In some embodiments, the salt is a trisuccinic acid salt.

In some embodiments, the composition contains at least about 50%, at least about 70%, at least about 80%, at least about 90%, at least about 95%, at least about 97%, at least about 98.0%, at least about 98.1%, at least about 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9% by weight of 6-methoxy-8-[4-(1-(5-fluoro)-quinolin-8-yl-piperidin-4-yl)-piperazin-1-yl]-quinoline trisuccinate as Form A, Form B, Form C or Form D.

In some embodiments, the composition is a pharmaceutical composition which contains at least one salt or crystalline form of the invention and at least one pharmaceutically acceptable carrier. In further embodiments, the composition is a pharmaceutical composition which contains at least one active pharmaceutical ingredient which is 6-methoxy-8-[4-(1-(5-fluoro)-quinolin-8-yl-piperidin-4-yl)-piperazin-1-yl]-quinoline trisuccininc acid salt or hydrate thereof, and at least one pharmaceutically acceptable carrier. In some embodiments, the pharmaceutical composition is suitable for oral administration. In some embodiments, the composition is provided in the form of a sustained release dosage form.

Pharmaceutically acceptable excipients (carriers) can be liquids, such as water and oils, including those of petroleum, animal, vegetable, or synthetic origin, such as peanut oil, soybean oil, mineral oil, sesame oil and the like. The excipients can be saline, gum acacia, gelatin, starch paste, talc, keratin, colloidal silica, urea and the like. In addition, auxiliary, stabilizing, thickening, lubricating, and coloring agents can be used. In one embodiment the excipients are sterile when administered to an animal. The excipient should be stable under the conditions of manufacture and storage and should be preserved against the contaminating action of microorganisms. Water is a particularly useful excipient when the compound or a pharmaceutically acceptable salt of the compound is administered intravenously. Saline solutions and aqueous dextrose and glycerol solutions can also be employed as liquid excipients, particularly for injectable solutions. Excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Liquid carriers can be used in preparing solutions, suspensions, emulsions, syrups, and elixirs. The salts and crystalline forms of this invention can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained release formulations, suppositories, emulsions, aerosols, sprays, suspensions, or any other form suitable for use. In one embodiment, the composition is in the form of a capsule. Other examples of suitable excipients are described in Remington's Pharmaceutical Sciences 1447 1676 (Alfonso R. Gennaro, ed., 19th ed. 1995).

In one embodiment, the salts and crystalline forms of the invention are formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided compound or pharmaceutically acceptable salt of the compound. In tablets, the compound or pharmaceutically acceptable salt of the compound is mixed with a carrier having the necessary compression properties in suitable proportions and compacted in the shape and size desired. The powders and tablets can contain up to about 99% of the salt or crystalline form.

Capsules may contain mixtures of the compounds or pharmaceutically acceptable salts of the compounds with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins.) Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

When in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the salts and crystalline forms can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the salts and crystalline forms are to be administered by infusion, they can be dispensed, for example, with an infusion bottle containing sterile pharmaceutical grade water or saline. Where the salts and crystalline forms are administered by injection, an ampule of sterile water for injection or saline can be provided so that the ingredients can be mixed prior to administration.

In another embodiment, the salts and crystalline forms can be administered transdermally through the use of a transdermal patch. Transdermal administrations include administrations across the surface of the body and the inner linings of the bodily passages including epithelial and mucosal tissues. Such administrations can be carried out using the present salts and crystalline forms in lotions, creams, foams, patches, suspensions, solutions, and suppositories (e.g., rectal or vaginal).

Transdermal administration can be accomplished through the use of a transdermal patch containing the salt or crystalline form of the invention and a carrier that is inert to the compound or pharmaceutically acceptable salt of the compound, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the compound or pharmaceutically acceptable salt of the compound into the blood stream, such as a semi-permeable membrane covering a reservoir containing the compound or pharmaceutically acceptable salt of the compound with or without a carrier, or a matrix containing the active ingredient.

The salts and crystalline forms of the invention may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The salts and crystalline forms can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of the salt or crystalline form to treat or prevent a 5-HT_{1A}-related disorder in a minimal amount of time. Advantages of controlled- or sustained-release compositions include extended activity of the drug, reduced dosage frequency, and increased compliance by the animal being treated. In addition, controlled or sustained release compositions can favorably affect the time of onset of action or other characteristics, such as blood levels of the compound or a pharmaceutically acceptable salt of the compound, and can thus reduce the occurrence of adverse side effects.

Controlled- or sustained-release compositions can initially release an amount of the compound that promptly produces the desired therapeutic or prophylactic effect, and gradually and continually release other amounts of the compound this level of therapeutic or prophylactic effect over an extended period of time. To maintain a constant level of the compound or a pharmaceutically acceptable salt of the compound in the body, the compound or a pharmaceutically acceptable salt of the compound can be released from the dosage form at a rate that will replace the amount of the compound or a pharmaceutically acceptable salt of the compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

The amount of the salt or crystalline form delivered is an amount that is effective for treating or preventing a 5-HT_{1A}-related disorder. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound is administered, the effective dosage amounts correspond to the total amount administered.

The amount of the salt or crystalline form that is effective for treating or preventing a 5-HT_{1A}-related disorder will typically range from about 0.001 mg/kg to about 600 mg/kg of body weight per day, in one embodiment, from about 1 mg/kg to about 600 mg/kg body weight per day, in another embodiment, from about 10 mg/kg to about 400 mg/kg body weight per day, in another embodiment, from about 10 mg/kg to about 200 mg/kg of body weight per day, in another embodiment, from about 10 mg/kg to about 100 mg/kg of body weight per day, in another embodiment, from about 1 mg/kg to about 10 mg/kg body weight per day, in another embodiment, from about 0.001 mg/kg to about 100 mg/kg of body weight per day, in another embodiment, from about 0.001 mg/kg to about 10 mg/kg of body weight per day, and in another embodiment, from about 0.001 mg/kg to about 1 mg/kg of body weight per day.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 0.01 mg/kg to about 250 mg/kg, and may be given in a single dose or in two or more divided doses. Variations in the dosage will necessarily occur depending upon the species, weight and condition of the patient being treated and the patient's individual response to the medicament.

In one embodiment, the unit dosage form is about 0.01 to about 1000 mg. In another embodiment, the unit dosage form is about 0.01 to about 500 mg; in another embodiment, the unit dosage form is about 0.01 to about 250 mg; in another embodiment, the unit dosage form is about 0.01 to about 100 mg; in another embodiment, the unit dosage form is about 0.01 to about 50 mg; in another embodiment, the unit dosage form is about 0.01 to about 25 mg; in another embodiment, the unit dosage form is about 0.01 to about 10 mg; in another embodiment, the unit dosage form is about 0.01 to about 5 mg; and in another embodiment, the unit dosage form is about 0.01 to about 10 mg.

In some embodiments, the composition is suitable for oral administration and/or comprises an oral dosage form.

The salts and crystalline forms can be assayed in vitro or in vivo for the desired therapeutic or prophylactic activity prior to use in humans. Animal model systems can be used to demonstrate safety and efficacy.

Pharmaceutical compositions can be prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those carriers that are compatible with the other ingredients in the formulation and are biologically acceptable.

Pharmaceutical Methods

The salts and crystalline forms of the invention are 5-HT_{1A} modulators which are useful in methods of treating various 5-HT_{1A}-related diseases or disorders such as cognition-related disorders or anxiety-related disorders.

Cognitive-related disorders can include improving cognitive function or inhibiting cognitive deficits. Examples of improvements in cognitive function include, without limitation, memory improvement and retention of learned information. Accordingly, the compounds are useful for slowing the loss of memory and cognition and for maintaining independent function for patients afflicted with a cognition-related disorder. Accordingly, the salts and crystalline forms of the present invention are useful for improving cognitive function. Further examples of cognition-related disorders include dementia, Parkinson's disease, Huntington's disease, Alzheimer's disease, cognitive deficits associated with Alzheimer's disease, mild cognitive impairment, and schizophrenia.

Example of anxiety-related disorders include attention deficit disorder, obsessive compulsive disorder, substance addiction, withdrawal from substance addiction, premenstrual dysphoric disorder, social anxiety disorder, anorexia nervosa, and bulimia nervosa.

The salts and crystalline forms of the invention are further useful for treating Alzheimer's disease. In some embodiments, the method for treating Alzheimer's disease includes administering a second therapeutic agent. In some embodiments, the second therapeutic agent is an anti-depressant agent, an anti-anxiety agent, an anti-psychotic agent, or a cognitive enhancer.

The salts and crystalline forms of the invention are further useful for treating mild cognitive impairment (MCI). In some embodiments, the method for treating MCI includes administering a second therapeutic agent. In some embodiments, the second therapeutic agent is an anti-depressant agent, an anti-anxiety agent, an anti-psychotic agent, or a cognitive enhancer.

The salts and crystalline forms of the invention are further useful for treating depression. In some embodiments, the method for treating depression includes administering a second therapeutic agent. In some embodiments, the second therapeutic agent is an anti-depressant agent, an anti-anxiety agent, an anti-psychotic agent, or a cognitive enhancer.

The salts and crystalline forms of the invention are further useful for treating sexual dysfunction, such as sexual dysfunction associated with drug treatment (e.g., with an antidepressant, an antipsychotic, or an anticonvulsant).

In certain embodiments, the drug treatment associated with sexual dysfunction involves a selective serotonin reuptake inhibitor (SSRI) (for example, fluoxetine, citalopram, escitalopram oxalate, fluvoxamine maleate, paroxetine, or sertraline), a tricyclic antidepressant (for example, desipramine, amitriptyline, amoxipine, clomipramine, doxepin, imipramine, nortriptyline, protriptyline, trimipramine, dothiepin, butriptyline, iprindole, or lofepramine), an aminoketone class compound (for example, bupropion). In some embodiments, the drug is a monoamine oxidase inhibitor (MAOI) (for example, pheneizine, isocarboxazid, or tranylcypromine), a serotonin and norepinepherine reuptake inhibitor (SNRI) (for example, venlafaxine, nefazodone, milnacipran, duloxetine), a norepinephrine reuptake inhibitor (NRI) (for example, reboxetine), a partial 5-HT_{1A} agonist (for example, buspirone), a 5-HT2A receptor antagonist (for example, nefazodone), a typical antipsychotic drug, or an atypical antipsychotic drug. Examples of such antipsychotic drugs include aliphatic phethiazine, a piperazine phenothiazine, a butyrophenone, a substituted benzamide, and a thioxanthine. Additional examples of such drugs include haloperidol, olanzapine, clozapine, risperidone, pimozide, aripiprazol, and ziprasidone. In some cases, the drug is an anticonvulsant, e.g., phenobarbital, phenyloin, primidone, or carbamazepine. In some cases, the patient in need of treatment for sexual dysfunction is being treated with at least two drugs that are antidepressant drugs, antipsychotic drugs, anticonvulsant drugs, or a combination thereof.

In some embodiments of the invention, the sexual dysfunction comprises a deficiency in penile erection.

In some embodiments, the salts or crystalline forms are effective for ameliorating sexual dysfunction in an animal model of sexual dysfunction associated with drug treatment, for example, in an animal model of sexual dysfunction that is an antidepressant drug-induced model of sexual dysfunction.

The salts and crystalline forms of the invention are further useful for improving sexual function in a patient.

As used herein, the term “patient” refers to any animal, including mammals, preferably mice, rats, other rodents, rabbits, dogs, cats, swine, cattle, sheep, horses, or primates, and most preferably humans. In some embodiments, the patient is in need of treatment.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual that may be predisposed to the disease, condition or disorder but does not yet experience or display the pathology or symptomatology of the disease;

(2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and

(3) ameliorating the disease; for example, ameliorating a disease, condition or disorder in an individual that is experiencing or displaying the pathology or symptomatology of the disease, condition or disorder (i.e., reversing the pathology and/or symptomatology).

Administration, Compositions, and Dosage Forms

The salts and crystalline forms of the invention can be administered neat or as a component of a composition that comprises a physiologically acceptable carrier or vehicle. A pharmaceutical composition of the invention can be prepared using a method comprising admixing the compound or a pharmaceutically acceptable salt of the compound and a physiologically acceptable carrier, excipient, or diluent. Admixing can be accomplished using methods well known for admixing a compound or a pharmaceutically acceptable salt of the compound and a physiologically acceptable carrier, excipient, or diluent.

The present pharmaceutical compositions can be administered orally. The salts and crystalline forms of the invention can also be administered by any other convenient route, for example, by infusion or bolus injection, by absorption through epithelial or mucocutaneous linings (e.g., oral, rectal, vaginal, and intestinal mucosa, etc.) and can be administered together with another therapeutic agent. Administration can be systemic or local. Various known delivery systems, including encapsulation in liposomes, microparticles, microcapsules, and capsules, can be used.

Methods of administration include, but are not limited to, intradermal, intramuscular, intraperitoneal, intravenous, subcutaneous, intranasal, epidural, oral, sublingual, intracerebral intravaginal, transdermal, rectal, by inhalation, or topical, particularly to the ears, nose, eyes, or skin. In some instances, administration will result of release of the compound or a pharmaceutically acceptable salt of the compound into the bloodstream. The mode of administration is left to the discretion of the practitioner.

In one embodiment, the salts and crystalline forms of the invention are administered orally.

In another embodiment, the salts and crystalline forms of the invention are administered intravenously.

In another embodiment, it may be desirable to administer the salts and crystalline forms of the invention locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, it can be desirable to introduce the salts and crystalline forms of the invention into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the salts and crystalline forms can be formulated as a suppository, with traditional binders and excipients such as triglycerides.

In another embodiment, the salts and crystalline forms of the invention can be delivered in a vesicle, in particular a liposome (see Langer, Science 249:1527-1533 (1990) and Treat et al., Liposomes in the Therapy of Infectious Disease and Cancer 317-327 and 353-365 (1989)).

In yet another embodiment, the salts and crystalline forms of the invention can be delivered in a controlled-release system or sustained-release system (see, e.g., Goodson, in Medical Applications of Controlled Release, vol. 2, pp. 115 138 (1984)). Other controlled or sustained-release systems discussed in the review by Langer, Science 249:1527 1533 (1990) can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

Combination Therapy

The salts and crystalline forms of the invention can be administered to a patient in combination with a therapeutically effective amount of one or more further therapeutic agents. Effective amounts of further therapeutic agents are well known to those skilled in the art. It is well within the skilled artisan's purview to determine the other therapeutic agent's optimal effective amount range. The salt or crystalline form and the other therapeutic agent can act additively or, in one embodiment, synergistically. In one embodiment of the invention, where another therapeutic agent is administered to an animal, the effective amount of salt or crystalline form is less than its effective amount would be where the other therapeutic agent is not administered. In this case, without being bound by theory, it is believed that the salt or crystalline form and the other therapeutic agent can act synergistically. In some cases, the patient in need of treatment is being treated with one or more other therapeutic agents. In some cases, the patient in need of treatment is being treated with at least two other therapeutic agents.

In one embodiment, the other therapeutic agent is selected from one or more of the following: anti-depressant agents, anti-anxiety agents, anti-psychotic agents, or cognitive enhancers. Examples of classes of antidepressants that can be used in combination with the active compounds of this invention include norepinephrine reuptake inhibitors, selective serotonin reuptake inhibitors (SSRIs), NK-1 receptor antagonists, monoamine oxidase inhibitors (MAOs), reversible inhibitors of monoamine oxidase (RIMAs), serotonin and noradrenaline reuptake inhibitors (SNRIs), corticotropin releasing factor (CRF) antagonists, α-adrenoreceptor antagonists, and atypical antidepressants. Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butriptyline, iprindole, lofepramine, nortriptyline, protriptyline, amoxapine, desipramine and maprotiline. Suitable selective serotonin reuptake inhibitors include fluoxetine, citolopram, escitalopram, fluvoxamine, paroxetine and sertraline. Examples of monoamine oxidase inhibitors include isocarboxazid, phenelzine, and tranylcypromine. Suitable reversible inhibitors of monoamine oxidase include moclobemide. Suitable serotonin and noradrenaline reuptake inhibitors of use in the present invention include venlafaxine, nefazodone, milnacipran, and duloxetine. Suitable CRF antagonists include those compounds described in International Patent Publication Nos. WO 94/13643, WO 94/13644, WO 94/13661, WO 94/13676 and WO 94/13677. Suitable atypical anti-depressants include bupropion, lithium, nefazodone, trazodone and viloxazine. Suitable NK-1 receptor antagonists include those referred to in International Patent Publication WO 01/77100.

Anti-anxiety agents that can be used in combination with the active compounds of this invention include without limitation benzodiazepines and serotonin 1A (5-HT_{1A}) agonists or antagonists, especially 5-HT_{1A} partial agonists, and corticotropin releasing factor (CRF) antagonists. Exemplary suitable benzodiazepines include alprazolam, chlordiazepoxide, clonazepam, chlorazepate, diazepam, halazepam, lorazepam, oxazepam, and prazepam. Exemplary suitable 5-HT_{1A} receptor agonists or antagonists include buspirone, flesinoxan, gepirone and ipsapirone.

Anti-psychotic agents that can be used in combination with the active compounds of this invention include without limitation aliphatic phethiazine, a piperazine phenothiazine, a butyrophenone, a substituted benzamide, and a thioxanthine. Additional examples of such drugs include without limitation haloperidol, olanzapine, clozapine, risperidone, pimozide, aripiprazol, and ziprasidone. In some cases, the drug is an anticonvulsant, e.g., phenobarbital, phenyloin, primidone, or carbamazepine.

Cognitive enhancers that can be used in combination with the active compounds of this invention include, without limitation, drugs that modulate neurotransmitter levels (e.g., acetylcholinesterase or cholinesterase inhibitors, cholinergic receptor agonists or serotonin receptor antagonists), drugs that modulate the level of soluble Aβ, amyloid fibril formation, or amyloid plaque burden (e.g., γ-secretase inhibitors, β-secretase inhibitors, antibody therapies, and degradative enzymes), and drugs that protect neuronal integrity (e.g., antioxidants, kinase inhibitors, caspase inhibitors, and hormones). Other representative candidate drugs that are co-administered with the compounds of the invention include cholinesterase inhibitors, (e.g., tacrine (COGNEX®), donepezil (ARICEPT®), rivastigmine (EXELON®) galantamine (REMINYL®), metrifonate, physostigmine, and Huperzine A), N-methyl-D-aspartate (NMDA) antagonists and agonists (e.g., dextromethorphan, memantine, dizocilpine maleate (MK-801), xenon, remacemide, eliprodil, amantadine, D-cycloserine, felbamate, ifenprodil, CP-101606 (Pfizer), Delucemine, and compounds described in U.S. Pat. Nos. 6,821,985 and 6,635,270), ampakines (e.g., cyclothiazide, aniracetam, CX-516 (Ampalex®), CX-717, CX-516, CX-614, and CX-691 (Cortex Pharmaceuticals, Inc. Irvine, Calif.), 7-chloro-3-methyl-3-4-dihydro-2H-1,2,4-benzothiadiazine S,S-dioxide (see Zivkovic et al., 1995, J. Pharmacol. Exp. Therap., 272:300-309; Thompson et al., 1995, Proc. Natl. Acad. Sci. USA, 92:7667-7671), 3-bicyclo[2,2,1]hept-5-en-2-yl-6-chloro-3,4-dihydro-2H-1,2,4-benzothiadiazine-7-sulfonamide-1,1-dioxide (Yamada, et al., 1993, J. Neurosc. 13:3904-3915); 7-fluoro-3-methyl-5-ethyl-1,2,4-benzothiadiazine-S,S-dioxide; and compounds described in U.S. Pat. No. 6,620,808 and International Patent Application Nos. WO 94/02475, WO 96/38414, WO 97/36907, WO 99/51240, and WO 99/42456), benzodiazepine (BZD)/GABA receptor complex modulators (e.g., progabide, gengabine, zaleplon, and compounds described in U.S. Pat. Nos. 5,538,956, 5,260,331, and 5,422,355); serotonin antagonists (e.g., 5HT receptor modulators, 5HT_{1A} antagonists or agonists (including without limitation lecozotan and compounds described in U.S. Pat. Nos. 6,465,482, 6,127,357, 6,469,007, and 6,586,436, and in PCT Publication No. WO 97/03982) and 5-HT₆ antagonists (including without limitation compounds described in U.S. Pat. Nos. 6,727,236, 6,825,212, 6,995,176, and 7,041,695)); nicotinics (e.g., niacin); muscarinics (e.g., xanomeline, CDD-0102, cevimeline, talsaclidine, oxybutin, tolterodine, propiverine, tropsium chloride and darifenacin); monoamine oxidase type B (MAO B) inhibitors (e.g., rasagiline, selegiline, deprenyl, lazabemide, safinamide, clorgyline, pargyline, N-(2-aminoethyl)-4-chlorobenzamide hydrochloride, and N-(2-aminoethyl)-5(3-fluorophenyl)-4-thiazolecarboxamide hydrochloride); phosphodiesterase (PDE) IV inhibitors (e.g., roflumilast, arofylline, cilomilast, rolipram, RO-20-1724, theophylline, denbufylline, ARIFLO, ROFLUMILAST, CDP-840 (a tri-aryl ethane) CP80633 (a pyrimidone), RP 73401 (Rhone-Poulenc Rorer), denbufylline (SmithKline Beecham), arofylline (Almirall), CP-77,059 (Pfizer), pyrid[2,3d]pyridazin-5-ones (Syntex), EP-685479 (Bayer), T-440 (Tanabe Seiyaku), and SDZ-ISQ-844 (Novartis)); G proteins; channel modulators; immunotherapeutics (e.g., compounds described in U.S. Patent Application Publication No. US 2005/0197356 and US 2005/0197379); anti-amyloid or amyloid lowering agents (e.g., bapineuzumab and compounds described in U.S. Pat. No. 6,878,742 or U.S. Patent Application Publication Nos. US 2005/0282825 or US 2005/0282826); statins and peroxisome proliferators activated receptor (PPARS) modulators (e.g., gemfibrozil (LOPID®), fenofibrate (TRICOR®), rosiglitazone maleate (AVANDIA®), pioglitazone (Actos™), rosiglitazone (Avandia™), clofibrate and bezafibrate); cysteinyl protease inhibitors; an inhibitor of receptor for advanced glycation endproduct (RAGE) (e.g., aminoguanidine, pyridoxaminem carnosine, phenazinediamine, OPB-9195, and tenilsetam); direct or indirect neurotropic agents (e.g., Cerebrolysin®, piracetam, oxiracetam, AIT-082 (Emilieu, 2000, Arch. Neurol. 57:454)); beta-secretase (BACE) inhibitors, α-secretase, immunophilins, caspase-3 inhibitors, Src kinase inhibitors, tissue plasminogen activator (TPA) activators, AMPA (alpha-amino-3-hydroxy-5-methyl-4-isoxazolepropionic acid) modulators, M4 agonists, JNK3 inhibitors, LXR agonists, H3 antagonists, and angiotensin IV antagonists. Other cognition enhancers include, without limitation, acetyl-1-carnitine, citicholine, huperzine, DMAE (dimethylaminoethanol), Bacopa monneiri extract, Sage extract, L-alpha glyceryl phosphoryl choline, Ginko biloba and Ginko biloba extract, Vinpocetine, DHA, nootropics including Phenyltropin, Pikatropin (from Creative Compounds, LLC, Scott City, Mo.), besipirdine, linopirdine, sibopirdine, estrogen and estrogenic compounds, idebenone, T-588 (Toyama Chemical, Japan), and FK960 (Fujisawa Pharmaceutical Co. Ltd.). Compounds described in U.S. Pat. Nos. 5,219,857, 4,904,658, 4,624,954 and 4,665,183 are also useful as cognitive enhancers as described herein. Cognitive enhancers that act through one or more of the above mechanisms are also within the scope of this invention.

In one embodiment, the salt or crystalline form of the invention and cognitive enhancer act additively or, in one embodiment, synergistically. In one embodiment, where a cognitive enhancer and a salt or crystalline form of the invention are co-administered to an animal, the effective amount of the salt or crystalline form of the invention is less than its effective amount would be where the cognitive enhancer agent is not administered. In one embodiment, where a cognitive enhancer and a salt or crystalline form of the invention are co-administered to an animal, the effective amount of the cognitive enhancer is less than its effective amount would be where the salt or crystalline form of the invention is not administered. In one embodiment, a cognitive enhancer and a salt or crystalline form of the invention are co-administered to an animal in doses that are less than their effective amounts would be where they were no co-administered. In these cases, without being bound by theory, it is believed that the compound or a pharmaceutically acceptable salt of the compound and the cognitive enhancer act synergistically.

In one embodiment, the other therapeutic agent is an agent useful for treating Alzheimer's disease or conditions associate with Alzheimer's disease, such as dementia. Exemplary agents useful for treating Alzheimer's disease include, without limitation, donepezil, rivastigmine, galantamine, memantine, and tacrine.

In one embodiment, the salt or crystalline form is administered concurrently with at least one further therapeutic agent.

In one embodiment, a composition comprising an effective amount of the salt or crystalline form and an effective amount of at least one further therapeutic agent within the same composition can be administered.

In another embodiment, a composition comprising an effective amount of the salt or crystalline form and a separate composition comprising an effective amount of a further therapeutic agent can be concurrently administered. In another embodiment, an effective amount of the salt or crystalline form is administered prior to or subsequent to administration of an effective amount of a further therapeutic agent. In this embodiment, the salt or crystalline form is administered while the other therapeutic agent exerts its therapeutic effect, or the other therapeutic agent is administered while the salt or crystalline form exerts its preventative or therapeutic effect for treating or preventing a 5-HT_{1A}-related disorder.

In order that the invention disclosed herein may be more efficiently understood, examples are provided below. It should be understood that these examples are for illustrative purposes only and are not to be construed as limiting the invention in any manner.

### EXAMPLES

### Example 1

### Preparation of 6-methoxy-8-[4-(1-(5-fluoro)-quinolin-8-yl-piperidin-4-yl)-piperazin-1-yl]-quinoline (free base)

### Step 1: 6-Methoxy-8-(1-piperazinyl)quinoline

A mixture of 8-amino-6-methoxyquinoline (150.0 g, 0.862 mol) and bis(2-chloroethyl)amine (219 g, 1.23 mol) in 6 parts (volume of hexanol v. weight of 8-amino-6-methoxyquinoline) of 1-hexanol (900 mL) was heated to 145° C. and stirred for 21 hours. Upon completion, the reaction mixture was cooled to 50-60° C. and 507 g of aqueous NaOH solution was added slowly. The reaction mixture was cooled to 25-30° C. and isopropyl acetate (750 mL) was added. The mixture was clarified through a Celite pad. The aqueous phase was then split off. The organic solution was treated with a slurry of adipic acid (126 g, 0.862 mol) in isopropyl acetate (250 mL). The resulting mixture was stirred for 16 hours to form 6-methoxy-8-(1-piperazinyl)quinoline adipate salt. The adipate salt was filtered and washed with isopropyl acetate (2×150 mL) and dried by nitrogen flow to give adipate of 6-methoxy-8-piperazin-1-yl-quinoline (186 g, 55% yield) with 97% HPLC area, 88% strength purity in 51% yield.

For purification of the salt, 580 g of the crude adipate salt and 2.8 L of methanol were mixed and heated to 65° C. and a dark solution was obtained. To this solution was charged slowly 1.1 liter of isopropyl acetate over 40 min at about 63° C. The mixture was stirred at about 63° C. for about 1 h and cooled to 0-5° C. After stirring at 0-5° C. for 2 hours, the mixture was filtered and washed with 300 mL of isopropyl acetate and dried with airflow. Yield, 395 g, 68.1% recovery yield.

To liberate 6-methoxy-8-(1-piperazinyl)quinoline from its adipate salt, 100 g (0.257 mol) of the adipate salt was added into a 2-L reactor followed by the addition of 500 mL of dichloromethane. To this mixture was added 100 g of water followed by the slow (in about 15 min) addition of 41 g of 50% sodium hydroxide solution to maintain the pH in the 13-14 range, adding sodium hydroxide solution as necessary if the pH is below 10. The organic bottom layer was separated and filtered through a pad of activated basic aluminum oxide (100 g, 6.5 cm diameter×3 cm depth). The pad was washed with 100 mL of isopropyl acetate twice. The dichloromethane was replaced by toluene by distillation under vacuum (450 to 500 mm Hg) while 3×150 mL of toluene was added into the reactor until the final volume was about 135 mL. Some white solid precipitated after distillation, the solid was removed by filtration, the filter cake was washed with 50 mL of toluene. Final volume, 185 mL, purity 97.56%, solution strength 27.4%).

### Step 2: 8-Bromo-5-fluoroquinoline

A 2-L reactor equipped with a mechanic agitator, a condenser, a thermocouple, a baffle, and nitrogen inlet was charged with 228 g of water, 200 g of 2-bromo-5-fluoroaniline and 80 g of 4-nitrophenol. To this mixture was charged 96% sulfuric acid in 10-30 min at 20-120° C. The mixture was heated to 135-140° C. and 194 g of glycerol was charged into the reactor over two hours at 135-145° C. The mixture was held at 135-145° C. for 1 hour after the addition. The reaction mixture was cooled to below 20-50° C. and slowly transferred to a 5-L reactor containing 1100 g of water and 1210 g of toluene. The 2-L reactor was washed with 300 g of water and the wash was combined into the 5-L reactor. The pH of the contents in the 5-L reactor was adjusted to pH 8-10 by adding approximately 1233 g (1370 mL) ammonium hydroxide (28-30% NH₃) at 20-40° C. The mixture was stirred at room temperature for 15 min and the solid by-product was filtered off while the filtrate was retained. The filter cake was washed with 400 mL of toluene and the all the filtrate was combined and charged a 3-L reactor. About 500 ml of 8.5% KOH solution was charged into the 3-L reactor and stirred for 10 min and bottom aqueous layer was spit off. A second portion of 500 ml of 8.5% KOH solution was added and the mixture was stirred for 15 min and the bottom aqueous layer was split off. Water 500 mL was added and stirred for 15 min before the bottom aqueous layer was split off. The organic layer was heated to distill off about 100-200 mL of toluene to azeotropically remove water. A clear solution was obtained. Yield: about 178 g 8-bromo-5-fluoroquinoline, ˜75%.

Alternatively, 8-bromo-5-fluoroquinoline was prepared by adding a warm mixture containing 2-bromo-5-fluoroaniline (10 g, 1.0 eq), 4-nitrophenol (40 g, 0.54 eq), and glycerol (97 g, 2.0 eq) over 1.5 hours to sulfuric acid (267 mL) and water (114 mL) at 140-150° C. The initial mixture showed 37.8% 4-nitrophenol by relative HPLC area %. Samples showed 4.7% 4-nitrophenol immediately after adding 50% of mixed starting materials and 5.0% immediately after adding all of the materials. The yield upon workup was 87.5%, with total impurities 0.29%. Addition of less (0.46 eq, 34 g) 4-nitrophenol also successfully produced the intermediate of interest at acceptable yield.

### Step 3: 1-(5-Fluoroquinolin-8-yl)piperidin-4-one

A 5-L jacketed cylindrical reactor equipped with an impeller-style agitator, condenser, thermocouple, and vacuum/nitrogen inlet was charged 2-L, 15% toluene solution of 8-bromo-5-fluoroquinoline, 209 g of 1,4-dioxa-8-azaspiro[4.5]decane. Meanwhile in a 500-mL Erlenmeyer flask, a suspension of 16.5 g (26.5 mmol) ±[1,1′-binaphthalene]-2,2′-diylbis[diphenyl-phosphine, and 6.08 g (6.64 mmol) tris[μ-[(1,2-η:4,5-η)-(1E,4E)-1,5-diphenyl-1,4-pentadien-3-one]]dipalladium in 260 g of toluene was prepared. This freshly made suspension was charged into the 5-L reactor followed by a rinse of 170 g of toluene. 166 g sodium tert-butoxide was then charged into the reactor followed by a rinse with 430 g of toluene. The reactor was degassed by vacuum to less than 125 mmHg and then filled with nitrogen to atmosphere three times. The mixture was then heated to 50-60° C. and stirred for 1 h and then heat to 65-75° and stirred at this temperature for about 10 hours. The mixture was cooled to 40-50° C. and then quenched with 800 g of water. The lower aqueous layer was split off and the volume of the organic layer was reduced to about 1.5 L by vacuum distillation. To this residual was charged 2.28 kg of 20% sulfuric acid at 25-30° C. The mixture was stirred for an hour and was clarified by filtration and a bi-phase filtrate was obtained. The aqueous phase was split and retained. Toluene 870 g was added to the aqueous solution and the mixture was neutralized by slowly adding 770 g 50% sodium hydroxide solution. The lower aqueous layer was split off and extracted with 600 g of toluene. The organic layers were combined and the volume of the reaction was reduced to about 1 L by vacuum distillation. The residue was cooled to room temperature and 480 g of toluene was charged. The mixture was heated to 45-55° C. to form a clear solution, which was filtered through a celite/charcoal pad to remove palladium. The filtrate was concentrated by vacuum distillation to about 0.7 L and diluted with 620 g heptane, cooled to −15 to −5° C. to form a slurry. The solid was collected by filtration. The product was dried by air flow at room temperature. Typical yield is about 70%.

### Step 4: 6-Methoxy-8-[4-(1-(5-fluoro)-quinolin-8-yl-piperidin-4-yl)-piperazin-1-yl]-quinoline

Toluene (118 g), sodium triacetoxyborohydride (44.5 g) were mixed at 0° C. to room temperature. To this mixture was charged a premixed toluene solution of 6-methoxy-8-(1-piperazinyl)quinoline (Step 1, 160 g, 27.4 wt % in toluene) and 1-(5-fluoroquinolin-8-yl)piperidin-4-one (Step 3, 41 g). The resulting mixture was stirred for 2 to 3 hours at about 30° C. KOH solution (443 g 9% in water) was charged to quench the residual sodium triacetoxyborohydride. Heptane (118 g) was added to further precipitate the product. The product was then filtered and washed with ethanol (2×100 ml). Yield 68 g, 86%. This crude product (67 g) was dissolved in 586 g dichloromethane and passed through a charcoal/celite pad to remove palladium. The dichloromethane was distilled off while 400 g of ethanol was slowly added at the same time. The resulting slurry was filtered and washed with ethanol twice (65 g+100 g). The product was dried in oven at 55° C. overnight. Purification recovery yield 59.9 g, 89.4%.

### Example 2

### Preparation of 6-methoxy-8-[4-(1-(5-fluoro)-quinolin-8-yl-piperidin-4-yl)-piperazin-1-yl]-quinoline trisuccinate salt (Form A: Method 1)

6-Methoxy-8-[4-(1-(5-fluoro)-quinolin-8-yl-piperidin-4-yl)-piperazin-1-yl]-quinoline (1.5089 g) was slurried in 100 mL of dichloromethane (99.8% HPLC) to obtain a clear light yellow solution of free base. Succinic acid (99%; 258.6 mg) was dissolved in 17 mL of acetone (99% HPLC). Then 15.275 mL of the succinic acid solution was added to 20 mL of the free base solution slowly. No immediate precipitation was observed. The resulting solution was allowed to evaporate to dryness at room temperature. The solid was analyzed by powder X-ray diffraction and found to be crystalline having Form A.

### Example 3

### Preparation of 6-methoxy-8-[4-(1-(5-fluoro)-quinolin-8-yl-piperidin-4-yl)-piperazin-1-yl]-quinoline trisuccinate salt (Form A: Method 2)

6-Methoxy-8-[4-(1-(5-fluoro)-quinolin-8-yl-piperidin-4-yl)-piperazin-1-yl]-quinoline (1.5089 g) was added to 100 mL of dichloromethane (99.8% HPLC) to obtain a clear light yellow solution of free base. Succinic acid (99%; 258.6 mg) was dissolved in 17 mL of acetone (99% HPLC). Then, 10 mL of the free base solution was added into 10.265 mL of the succinic acid solution. No immediate precipitation was observed. The resulting solution turned turbid after stirring with a magnetic stirrer bar at room temperature for a short period of time. The resulting slurry was stirred overnight at room temperature and then suction-filtered using 0.2 μm filter paper. The off-white solid was analyzed by powder X-ray diffraction and found to be crystalline having Form A.

### Example 4

### Preparation of 6-methoxy-8-[4-(1-(5-fluoro)-quinolin-8-yl-piperidin-4-yl)-piperazin-1-yl]-quinoline trisuccinate salt (Form A: Method 3)

6-Methoxy-8-[4-(1-(5-fluoro)-quinolin-8-yl-piperidin-4-yl)-piperazin-1-yl]-quinoline (102.0 mg) was added to 5 mL of tetrahydrofuran (THF). The resulting slurry was heated to approximately 50° C. to obtain a clear solution. To a separate vial, 81.0 mg of succinic acid (99%) was slurried in 1.5 mL of THF. The acid slurry was sonicated for approximately 1 minute to obtain a clear solution. The acid solution was added into the free base solution. Then, 3 mL of heptane was added into 3.25 mL of the solution mixture as anti-solvent. Immediate precipitation was observed and the slurry was stirred in ice/water bath for approximately 2 hours and then filtered under vacuum using 0.2 μm filter paper. The solid was characterized by differential scanning calorimetry and powder X-ray diffraction which was consistent with Form A.

### Example 5

### Preparation of 6-methoxy-8-[4-(1-(5-fluoro)-quinolin-8-yl-piperidin-4-yl)-piperazin-1-yl]-quinoline trisuccinate salt (Form B)

6-Methoxy-8-[4-(1-(5-fluoro)-quinolin-8-yl-piperidin-4-yl)-piperazin-1-yl]-quinoline (102.0 mg) was added into 5 mL of THF. The slurry was heated to approximately 50° C. to obtain a clear solution of free base. To a separate vial, 81.0 mg of succinic acid (99%) was slurried in 1.5 mL of THF. The acid slurry was sonicated for approximately 1 minute to obtain a clear solution. The acid solution was added into the free base solution. Then 3.25 mL of the acid and base mixture was transferred to a separate vial and the vial was cooled in an ice/water bath. Then 3 mL of heptane was added to the mixture as anti-solvent. Immediate precipitation was observed. The slurry was removed from the ice/water bath and the solid was isolated by vacuum filtration. The solid was analyzed by powder X-ray diffraction and found to be crystalline, having Form B. This sample was further characterized by differential scanning calorimetry as described herein and appeared to be anhydrous. Form B was found to convert to Form A when slurried in a mixture of THF and heptane for about 2 hours in an ice/water bath.

### Example 6

### Preparation of 6-methoxy-8-[4-(1-(5-fluoro)-quinolin-8-yl-piperidin-4-yl)-piperazin-1-yl]-quinoline trisuccinate salt (Form C)

Water (HPLC; 20 mL) was added into 16.9 mg of trisuccinate Form A (see Example 2). The mixture was sonicated for approximate 1 minute to a turbid solution. The solution was slurried at room temperature for approximate 13 days. The solid was isolated from the turbid solution by vacuum filtration using 0.2 μm filter paper. The white solid was analyzed by powder X-ray diffraction and found to be crystalline, having Form C. This sample was further characterized by differential scanning calorimetry as described herein and appeared to be hydrated.

### Example 7

### Preparation of 6-methoxy-8-[4-(1-(5-fluoro)-quinolin-8-yl-piperidin-4-yl)-piperazin-1-yl]-quinoline trisuccinate salt (Form D; Method 1)

Distilled water (200 μL) was added to Form A. The resulting slurry was quickly mixed with a spatula and then vacuum filtered using a 0.2 μm filter paper. The solid was analyzed by powder X-ray diffraction and found to be crystalline, having Form D. This sample was further characterized by differential scanning calorimetry as described herein and appeared to be hydrated.

### Example 8

### Preparation of 6-methoxy-8-[4-(1-(5-fluoro)-quinolin-8-yl-piperidin-4-yl)-piperazin-1-yl]-quinoline trisuccinate salt (Form D; Method 2)

Water (HPLC, 1 mL) and 1 mL of ethanol (99%, HPLC) were added to 14.4 mg of trisuccinate Form A (see Example 3). The mixture was shaken to obtain a clear solution. The solution was filtered using a syringe fitted with a 0.2 μm filter tip and allowed to evaporate to dryness at room temperature. The recovered light yellow powder was analyzed by powder X-ray diffraction and found to be crystalline, having Form D. The solid was also characterized by differential scanning calorimetry.

### Example 9

### ¹H NMR characterization of 6-methoxy-8-[4-(1-(5-fluoro)-quinolin-8-yl-piperidin-4-yl)-piperazin-1-yl]-quinoline trisuccinate salt

Proton nuclear magnetic resonance (¹H NMR) spectra were obtained on a Bruker Advance DRX-400 MHz NMR spectrometer, equipped with a 5 mm QNP probe. About 5 mg sample was dissolved in 0.6 mL DMSO-d₆ (99.9% D), containing 0.05% TMS as an internal reference. Stoichiometry of the salts was determined by integrating the peak area of succinate at 2.42 ppm and comparing it to the unique peak areas of 6-methoxy-8-[4-(1-(5-fluoro)-quinolin-8-yl-piperidin-4-yl)-piperazin-1-yl]-quinoline to obtain molar ratio of succinic acid to free base. The salts prepared according to Examples 2 and 3 were confirmed as trisuccinate salts. Percentages of counter ions were further confirmed, using molecular weight of 471.57 for free base and 118.09 for succinic acid, and were consistent with a trisuccinate salt. See Table 4 below.

TABLE 3

| | | Acid/free base | Wt % | |
|---|---|---|---|---|
| Sample ID | Counter Ion | Molar Ratio | Counter Ion | M.W. |
| Example 1 | Free base | N/A | NA | 471.57 |
| Example 2 | Trisuccinate | 2.9 | 42% | 825.87 |
| Example 3 | Trisuccinate | 2.9 | 42% | 825.87 |

### Example 10

### X-Ray Powder Diffraction (XRPD) Collection Parameters

X-ray powder diffraction patterns were obtained on a Rigaku Miniflex Diffraction System (Rigaku MSC inc.). The powder samples were deposited on a zero-background polished silicon sample holder. A normal focus copper x-ray tube at 0.45 kW equipped with a Ni Kβ filter scanning at 2 degrees/minute from 3.00 to 40.00 degree 2-theta was used as the X-ray source. The data processing was carried out using Jade 6.0 software.

### Example 11

### Differential Scanning Calorimetry (DSC) Parameters

DSC data was collected using a Q1000 DSC (TA instruments). Typically, 3-5 mg of sample was placed in a hermetically sealed aluminum pan (no pin-hole). The sample was heated from 40-250° C. at a ramp rate of 10° C./min. The heat flow data was analyzed using Universal Analysis software (TA instruments).

### Example 12

### Thermogravimetric Analysis (TGA) Parameters

TGA data was collected using a Q500 thermogravimetric analyzer (TA instruments). About 5-20 mg of sample was deposited on a clean platinum pan and heated from 25° C. to 300° C. at 10° C./min under a 40 mL/min N₂ flow. The data was analyzed using Universal Analysis software (TA instruments).

Various modifications of the invention, in addition to those described herein, will be apparent to those skilled in the art from the foregoing description. Such modifications are also intended to fall within the scope of the appended claims. Each reference, including all patents, patent applications, and journal literature, cited in the present application is incorporated herein by reference in its entirety.

What is claimed is:

1. A succinate salt of 6-methoxy-8-[4-(1-(5-fluoro)-quinolin-8-yl-piperidin-4-yl)-piperazin-1-yl]-quinoline.

2. The salt of claim 1 which is a trisuccinate salt.

3. The salt of claim 1 which is crystalline.

4. The salt of claim 1 which is anhydrous.

5. A crystalline form (Form A) of 6-methoxy-8-[4-(1-(5-fluoro)-quinolin-8-yl-piperidin-4-yl)-piperazin-1-yl]-quinoline trisuccinate having an X-ray powder diffraction pattern comprising characteristic peaks, in terms of 2θ (°), at about 8.1 and about 22.4.

6. The crystalline form of claim 5 further comprising a characteristic peak, in terms of 2θ (°), at about 10.2.

7. The crystalline form of claim 6 further comprising a characteristic peak, in terms of 2θ (°), at about 16.9.

8. A crystalline form (Form A) of 6-methoxy-8-[4-(1-(5-fluoro)-quinolin-8-yl-piperidin-4-yl)-piperazin-1-yl]-quinoline trisuccinate having an X-ray powder diffraction pattern comprising a characteristic peak, in terms of 2θ, of about 8.1 and at least three characteristic peaks, in terms of 2θ (°), selected from about 7.3, about 10.2, about 16.9, about 17.3, about 17.7, about 22.4, about 23.2, about 26.5, about 27.3, and about 29.7.

9. A crystalline form (Form A) of 6-methoxy-8-[4-(1-(5-fluoro)-quinolin-8-yl-piperidin-4-yl)-piperazin-1-yl]-quinoline trisuccinate having an X-ray powder diffraction pattern substantially as shown in FIG. 1.

10. The crystalline form of claim 5 having a DSC thermogram which is characterized by an endothermic peak at about 179° C.

11. The crystalline form of claim 5 having a DSC thermogram substantially as shown in FIG. 5.

12. The crystalline form of claim 5 having a TGA profile substantially as shown in FIG.

13. A crystalline form (Form B) of 6-methoxy-8-[4-(1-(5-fluoro)-quinolin-8-yl-piperidin-4-yl)-piperazin-1-yl]-quinoline trisuccinate having an X-ray powder diffraction pattern comprising characteristic peaks, in terms of 2θ (°), at about 7.1 and about 21.0.

14. The crystalline form of claim 13 further comprising a characteristic peak, in terms of 2θ (°), at about 15.5.

15. The crystalline form of claim 14 further comprising a characteristic peak, in terms of 2θ (°), at about 25.9.

16. A crystalline form (Form B) of 6-methoxy-8-[4-(1-(5-fluoro)-quinolin-8-yl-piperidin-4-yl)-piperazin-1-yl]-quinoline trisuccinate having an X-ray powder diffraction pattern comprising a characteristic peak, in terms of 2θ (°), of about 7.1 and at least three characteristic peaks, in terms of 2θ, selected from about 8.7, about 14.5, about 15.5, about 16.1, about 17.9, about 19.3, about 21.0, about 23.3, about 24.0, and about 25.9.

17. A crystalline form (Form B) of 6-methoxy-8-[4-(1-(5-fluoro)-quinolin-8-yl-piperidin-4-yl)-piperazin-1-yl]-quinoline trisuccinate having an X-ray powder diffraction pattern substantially as shown in FIG. 2.

18. The crystalline form of claim 13 having a DSC thermogram substantially as shown in FIG. 6.

19. A crystalline form (Form C) of 6-methoxy-8-[4-(1-(5-fluoro)-quinolin-8-yl-piperidin-4-yl)-piperazin-1-yl]-quinoline trisuccinate having an X-ray powder diffraction pattern comprising characteristic peaks, in terms of 2θ (°), at about 8.0 and about 10.7.

20. The crystalline form of claim 19 further comprising a characteristic peak, in terms of 2θ (°), at about 16.1.

21. The crystalline form of claim 20 further comprising a characteristic peak, in terms of 2θ (°), at about 23.1.

22. A crystalline form (Form C) of 6-methoxy-8-[4-(1-(5-fluoro)-quinolin-8-yl-piperidin-4-yl)-piperazin-1-yl]-quinoline trisuccinate having an X-ray powder diffraction pattern comprising a characteristic peak, in terms of 2θ, of about 10.7 and at least three characteristic peaks, in terms of 2θ (°), selected from about 8.0, about 16.1, about 18.7, about 19.1, about 21.9, about 22.7, about 23.1, about 24.7, about 26.0, about 26.3, about 26.9, and about 32.5.

23. A crystalline form (Form C) of 6-methoxy-8-[4-(1-(5-fluoro)-quinolin-8-yl-piperidin-4-yl)-piperazin-1-yl]-quinoline trisuccinate having an X-ray powder diffraction pattern substantially as shown in FIG. 3.

24. The crystalline form of claim 19 having a DSC thermogram substantially as shown in FIG. 7.

25. A crystalline form (Form D) of 6-methoxy-8-[4-(1-(5-fluoro)-quinolin-8-yl-piperidin-4-yl)-piperazin-1-yl]-quinoline trisuccinate having an X-ray powder diffraction pattern comprising characteristic peaks, in terms of 2θ (°), at about 11.0 and about 27.3.

26. The crystalline form of claim 25 further comprising a characteristic peak, in terms of 2θ (°), at about 28.3.

27. The crystalline form of claim 26 further comprising a characteristic peak, in terms of 2θ (°), at about 20.7.

28. A crystalline form (Form D) of 6-methoxy-8-[4-(1-(5-fluoro)-quinolin-8-yl-piperidin-4-yl)-piperazin-1-yl]-quinoline trisuccinate having an X-ray powder diffraction pattern comprising a characteristic peak, in terms of 2θ (°), of about 11.0 and at least three characteristic peaks, in terms of 2θ, selected from about 14.1, about 15.0, about 19.3, about 20.3, about 20.7, about 22.0, about 25.6, about 27.3, about 28.3, and about 32.3.

29. A crystalline form (Form D) of 6-methoxy-8-[4-(1-(5-fluoro)-quinolin-8-yl-piperidin-4-yl)-piperazin-1-yl]-quinoline trisuccinate having an X-ray powder diffraction pattern substantially as shown in FIG. 4.

30. The crystalline form of claim 25 having a DSC thermogram substantially as shown in FIG. 8.

31. The crystalline form of claim 25 having a TGA profile substantially as shown in FIG. 9.

32. A process for preparing the crystalline form (Form A) of claim 5, 8, or 9 comprising precipitating said crystalline form from a solution of 6-methoxy-8-[4-(1-(5-fluoro)-quinolin-8-yl-piperidin-4-yl)-piperazin-1-yl]-quinoline trisuccinate in organic solvent which is substantially free of water.

33. The process of claim 32 wherein said organic solvent comprises about 1% by volume of water or less.

34. The process of claim 32 wherein said organic solvent comprises dichloromethane, acetone, tetrahydrofuran, or combination thereof.

35. The process of claim 32 wherein said precipitating is induced by addition of antisolvent or reduction of temperature, or a combination of both.

36. A crystalline form prepared by the process of claim 32.

37. A process for preparing the crystalline form (Form B) of claim 13, 16, or 17 comprising precipitating said crystalline form from a solution of 6-methoxy-8-[4-(1-(5-fluoro)-quinolin-8-yl-piperidin-4-yl)-piperazin-1-yl]-quinoline trisuccinate in organic solvent which is substantially free of water.

38. The process of claim 37 wherein said organic solvent comprises about 1% by volume of water or less.

39. The process of claim 37 wherein said organic solvent comprises tetrahydrofuran.

40. The process of claim 37 wherein said precipitating is induced by addition of antisolvent and reduction of temperature.

41. The process of claim 37 further comprising isolating said crystalline form from the reaction mixture prior to its conversion to a different crystalline form.

42. A crystalline form prepared by the process of claim 37.

43. A process for preparing the crystalline form of claim 19, 22, or 23 comprising slurrying Form A in water.

44. A crystalline form prepared by the process of claim 43.

45. A process for preparing the crystalline form of claim 25, 28, or 29 comprising slurrying Form A in a mixture comprising water and an alcohol.

46. The process of claim 45 wherein said alcohol is ethanol.

47. A crystalline form prepared by the process of claim 45.

48. A method for treating a 5-HT_{1A}-related disorder in a patient in need thereof, the method comprising administering to said patient a therapeutically effective amount of a salt of claim 1 or 2 or a crystalline form of claim 5, 13, 19, or 25.

49. The method of claim 48 wherein the 5-HT_{1A}-related disorder is a cognition-related disorder or an anxiety-related disorder.

50. The method of claim 49 wherein the cognition-related disorder is dementia, Parkinson's disease, Huntington's disease, Alzheimer's disease, cognitive deficits associated with Alzheimer's disease, mild cognitive impairment, or schizophrenia.

51. The method of claim 49, wherein the anxiety-related disorder is attention deficit disorder, obsessive compulsive disorder, substance addiction, withdrawal from substance addiction, premenstrual dysphoric disorder, social anxiety disorder, anorexia nervosa, or bulimia nervosa.

52. The method of claim 49 further comprising administering a second therapeutic agent.

53. The method of claim 49 wherein the second therapeutic agent is an anti-depressant agent, an anti-anxiety agent, anti-psychotic agent, or a cognitive enhancer.

54. The method of claim 49 wherein the second therapeutic agent is a selective serotonin reuptake inhibitor.

55. The method of claim 49 wherein the second therapeutic agent is fluoxetine, fluvoxamine, paroxetine, sertaline, clonazepam, diazepam, buspirone, haloperidol, olanzapine, or clozapine.

56. The method of claim 49 wherein the second therapeutic agent is a cholinesterase inhibitor.

57. The method of claim 49 wherein the second therapeutic agent is tacrine, donepezil, rivastigmine, or galantamine.

58. A method for treating Alzheimer's disease in a patient in need thereof, the method comprising administering to said patient a therapeutically effective amount of a salt of claim 1 or 2 or a crystalline form of claim 5, 13, 19, or 25.

59. The method of claim 58 further comprising administering a second therapeutic agent.

60. The method of claim 59 wherein the second therapeutic agent is an anti-depressant agent, an anti-anxiety agent, anti-psychotic agent, or a cognitive enhancer.

61. The method of claim 59 wherein the second therapeutic agent is a selective serotonin reuptake inhibitor.

62. The method of claim 59 wherein the second therapeutic agent is fluoxetine, fluvoxamine, paroxetine, sertaline, clonazepam, diazepam, buspirone, haloperidol, olanzapine, or clozapine.

63. The method of claim 59 wherein the second therapeutic agent is a cholinesterase inhibitor.

64. The method of claim 59 wherein the second therapeutic agent is tacrine, donepezil, rivastigmine, or galantamine.

65. A method for treating mild cognitive impairment (MCI) in a patient in need thereof, the method comprising administering to said patient a therapeutically effective amount of a compound of a salt of claim 1 or 2 or a crystalline form of claim 5, 13, 19, or 25.

66. The method of claim 65 further comprising administering a second therapeutic agent.

67. The method of claim 66 wherein the second therapeutic agent is an anti-depressant agent, an anti-anxiety agent, anti-psychotic agent, or a cognitive enhancer.

68. The method of claim 66 wherein the second therapeutic agent is a selective serotonin reuptake inhibitor.

69. The method of claim 66 wherein the second therapeutic agent is fluoxetine, fluvoxamine, paroxetine, sertaline, clonazepam, diazepam, buspirone, haloperidol, olanzapine, or clozapine.

70. The method of claim 66 wherein the second therapeutic agent is a cholinesterase inhibitor.

71. The method of claim 66 wherein the second therapeutic agent is tacrine, donepezil, rivastigmine, or galantamine.

72. A method for treating depression in a patient in need thereof, the method comprising administering to said patient a therapeutically effective amount of a salt of claim 1 or 2, or a crystalline form of claim 5, 13, 19, or 25.

73. The method of claim 72 further comprising administering a second therapeutic agent.

74. The method of claim 73 wherein the second therapeutic agent is an anti-depressant agent, an anti-anxiety agent, anti-psychotic agent, or a cognitive enhancer.

75. The method of claim 73 wherein the second therapeutic agent is a selective serotonin reuptake inhibitor.

76. The method of claim 73 wherein the second therapeutic agent is fluoxetine, fluvoxamine, paroxetine, sertaline, clonazepam, diazepam, buspirone, haloperidol, olanzapine, or clozapine.

77. The method of claim 73 wherein the second therapeutic agent is a cholinesterase inhibitor.

78. The method of claim 73 wherein the second therapeutic agent is tacrine, donepezil, rivastigmine, or galantamine.

79. A method for treating sexual dysfunction associated with drug treatment in a patient in need thereof, the method comprising administering to the patient a therapeutically effective amount of a salt of claim 1 or 2 or a crystalline form of claim 5, 13, 19, or 25.

80. The method of claim 79 wherein the drug treatment is antidepressant drug treatment, antipsychotic drug treatment, or anticonvulsant drug treatment.

81. A method of improving sexual function in a patient in need thereof, the method comprising administering to the patient an effective amount of a salt of claim 1 or 2 or crystalline form of claim 5, 13, 19, or 25.

82. A composition comprising a salt of claim 1 or 2 or crystalline form of claim 5, 13, 19, or 25 and at least one pharmaceutically acceptable carrier.

83. The composition of claim 82 further comprising a second therapeutic agent.

83. The composition of claim 83 wherein said second therapeutic agent is an anti-depressant agent, an anti-anxiety agent, anti-psychotic agent, or a cognitive enhancer.

84. The composition of claim 83 wherein said second therapeutic agent is a selective serotonin reuptake inhibitor.

85. The composition of claim 83 wherein said second therapeutic agent is fluoxetine, fluvoxamine, paroxetine, sertaline, clonazepam, diazepam, buspirone, haloperidol, olanzapine, or clozapine.

86. The composition of claim 83 wherein said second therapeutic agent is a cholinesterase inhibitor.

87. The composition of claim 83 wherein said second therapeutic agent is tacrine, donepezil, rivastigmine, or galantamine.
